(19) United States
(12) Patent Application Publication
(10) Pub. No.: US 2005/0074863 A1
(43) Pub. Date: Apr. 7, 2005

(54) T-cell epitodes in carboxypeptidase g2

(76) Inventors: Koen Hellendoorn, Newmarket (GB); Matthew Baker, Cambridge (GB); Steven Williams, Abberdeenshire (GB); Francis J. Carr, Aberdeenshire (GB)

Correspondence Address: OLSON & HIERL, LTD., 20 NORTH WACKER DRIVE, 36TH FLOOR, CHICAGO, IL, 60606, US

(21) Appl. No.: 10/497,091

(22) PCT Filed: Nov. 27, 2002
(86) PCT No.: PCT/EP02/13351

(30) Foreign Application Priority Data
Nov. 29, 2001 (EP) 01128519.4
Jan. 25, 2002 (EP) 02001778.6
Sep. 13, 2002 (EP) 02020634.8

(51) Int. Cl. C12N 9/48; C07H 21/04; C12N 15/74

(52) U.S. Cl. 435/212; 435/69.1; 435/252.3; 435/471; 536/23.2

(57) ABSTRACT

The invention in particular relates to the modification of a bacterial enzyme carboxypeptidease G2 (CPG2) to result in CPG2 proteins that are substantially non-immunogenic or less immunogenic than any non-modified counterpart when used in vivo. The present invention relates also to T-cell epitope peptides derived from said non-modified protein by means of which it is possible to create modified CPG2 variants with reduced immunogenicity. These polypeptides are suitable particularly for therapeutic use in humans.

### FIELD OF THE INVENTION

The present invention relates to polypeptides to be administered especially to humans and in particular for therapeutic use. The polypeptides are modified polypeptides whereby the modification results in a reduced propensity for the polypeptide to elicit an immune response upon administration to the human subject. The invention in particular relates to the modification of a bacterial enzyme carboxypeptidease G2 (CPG2) to result in CPG2 proteins that are substantially non-immunogenic or less immunogenic than any non-modified counterpart when used in vivo. The invention relates furthermore to T-cell epitope peptides derived from said non-modified protein by means of which it is possible to create modified CPG2 variants with reduced immunogenicity.

### BACKGROUND OF THE INVENTION

There are many instances whereby the efficacy of a therapeutic protein is limited by an unwanted immune reaction to the therapeutic protein. Several mouse monoclonal antibodies have shown promise as therapies in a number of human disease settings but in certain cases have failed due to the induction of significant degrees of a human anti-murine antibody (HAMA) response [Schroff, R. W. et al (1985) Cancer Res. 45: 879-885; Shawler, D. L. et al (1985) J. Immunol. 135: 1530-1535]. For monoclonal antibodies, a number of techniques have been developed in attempt to reduce the HAMA response [WO 89/09622; EP 0239400; EP 0438310; WO 91/06667]. These recombinant DNA approaches have generally reduced the mouse genetic information in the final antibody construct whilst increasing the human genetic information in the final construct.

Notwithstanding, the resultant “humanized” antibodies have, in several cases, still elicited an immune response in patients [Issacs J. D. (1990) Sem. Immunol. 2: 449, 456; Rebello, P. R. et al (1999) Transplantation 68: 1417-1420].

Antibodies are not the only class of polypeptide molecule administered as a therapeutic agent against which an immune response may be mounted. Even proteins of human origin and with the same amino acid sequences as occur within humans can still induce an immune response in humans. Notable examples amongst others include the therapeutic use of granulocyte-macrophage colony stimulating factor [Wadhwa, M. et al (1999) Clin. Cancer Res. 5: 1353-1361] and interferon alpha 2 [Russo, D. et al (1996) Bri. J. Haem. 94: 300-305; Stein, R. et al (1988) New Engl. J. Med. 318: 1409-1413]. In such situations where these human proteins are immunogenic, there is a presumed breakage of immunological tolerance that would otherwise have been operating in these subjects to these proteins.

This situation is different where the human protein is being administered as a replacement therapy for example in a genetic disease where there is a constitutional lack of the protein such as can be the case for diseases such as hemophilia A, Christmas disease, Gauchers disease and numerous other examples. In such cases, the therapeutic replacement protein may function immunologically as a foreign molecule from the outset, and where the individuals are able to mount an immune response to the therapeutic, the efficacy of the therapy is likely to be significantly compromised.

Irrespective of whether the protein therapeutic is seen by the host immune system as a foreign molecule, or if an existing tolerance to the molecule is overcome, the mechanism of immune reactivity to the protein is the same. Key to the induction of an immune response is the presence within the protein of peptides that can stimulate the activity of T-cells via presentation on MHC class II molecules, so-called “T-cell epitopes”. Such T-cell epitopes are commonly defined as any amino acid residue sequence with the ability to bind to MHC Class II molecules. Implicitly, a “T-cell epitope” means an epitope which when bound to MHC molecules can be recognized by a T-cell receptor (TCR), and which can, at least in principle, cause the activation of these T-cells by engaging a TCR to promote a T-cell response.

MHC Class II molecules are a group of highly polymorphic proteins which play a central role in helper T-cell selection and activation. The human leukocyte antigen group DR (HLA-DR) are the predominant isotype of this group of proteins however, isotypes HLA-DQ and HLA-DP perform similar functions. In the human population, individuals bear two to four DR alleles, two DQ and two DP alleles. The structure of a number of DR molecules has been solved and these appear as an open-ended peptide binding groove with a number of hydrophobic pockets which engage hydrophobic residues (pocket residues) of the peptide [Brown et al Nature (1993) 364: 33; Stem et al (1994) Nature 368: 215]. Polymorphism identifying the different allotypes of class II molecule contributes to a wide diversity of different binding surfaces for peptides within the peptide binding grove and at the population level ensures maximal flexibility with regard to the ability to recognize foreign proteins and mount an immune response to pathogenic organisms.

An immune response to a therapeutic protein proceeds via the MHC class II peptide presentation pathway. Here exogenous proteins are engulfed and processed for presentation in association with MHC class II molecules of the DR, DQ or DP type. MHC Class II molecules are expressed by professional antigen presenting cells (APCs), such as macrophages and dendritic cells amongst others. Engagement of a MHC class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

T-cell epitope identification is the first step to epitope elimination, however there are few clear cases in the art where epitope identification and epitope removal are integrated into a single scheme. Thus WO98/52976 and WO00/34317 teach computational threading approaches to identifying polypeptide sequences with the potential to bind a sub-set of human MHC class II DR allotypes. In these teachings, predicted T-cell epitopes are removed by the use of judicious amino acid substitution within the protein of interest. However with this scheme and other computationally based procedures for epitope identification [Godkin, A. J. et al (1998) J. Immunol. 161: 850-858; Sturniolo, T. et al (1999) Nat. Biotechnol. 17: 555-561], peptides predicted to be able to bind MHC class II molecules may not function as T-cell epitopes in all situations, particularly, in vivo due to the processing pathways or other phenomena. In addition, the computational approaches to T-cell epitope prediction have in general not been capable of predicting epitopes with DP or DQ restriction.

Equally, in vitro methods for measuring the ability of synthetic peptides to bind MHC class II molecules, for example using B-cell lines of defined MHC allotype as a source of MHC class II binding surface and may be applied to MHC class II ligand identification [Marshall K. W. et al. (1994) J. Immunol. 152:4946-4956; O'Sullivan et al (1990) J. Immunol. 145: 1799-1808; Robadey C. et al (1997) J. Immunol 159: 3238-3246].

However, such techniques are not adapted for the screening multiple potential epitopes to a wide diversity of MHC allotypes, nor can they confirm the ability of a binding peptide to function as a T-cell epitope.

Recently techniques exploiting soluble complexes of recombinant MHC molecules in combination with synthetic peptides have come into use [Kern, F. et al (1998) Nature Medicine 4:975-978; Kwok, W. W. et al (2001) TRENDS in Immunol. 22:583-588]. These reagents and procedures are used to identify the presence of T-cell clones from peripheral blood samples from human or experimental animal subjects that are able to bind particular MHC-peptide complexes and are not adapted for the screening multiple potential epitopes to a wide diversity of MHC allotypes.

Biological assays of T-cell activation provide a practical option to providing a reading of the ability of a test peptide/protein sequence to evoke an immune response. Examples of this kind of approach include the work of Petra et al using T-cell proliferation assays to the bacterial protein staphylokinase, followed by epitope mapping using synthetic peptides to stimulate T-cell lines [Petra, A. M. et al (2002) J. Immunol. 168: 155-161].

Similarly, T-cell proliferation assays using synthetic peptides of the tetanus toxin protein have resulted in definition of immunodominant epitope regions of the toxin [Reece J. C. et al (1993)J. Immunol. 151: 6175-6184]. WO99/53038 discloses an approach whereby T-cell epitopes in a test protein may be determined using isolated sub-sets of human immune cells, promoting their differentiation in vitro and culture of the cells in the presence of synthetic peptides of interest and measurement of any induced proliferation in the cultured T-cells. The same technique is also described by Stickler et al [Stickler, M. M. et al (2000) J. Immunotherapy 23:654-660], where in both instances the method is applied to the detection of T-cell epitopes within bacterial subtilisin. Such a technique requires careful application of cell isolation techniques and cell culture with multiple cytokine supplements to obtain the desired immune cell sub-sets (dendritic cells, CD4+ and or CD8+ T-cells) and is not conducive to rapid through-put screening using multiple donor samples.

As depicted above and as consequence thereof, it would be desirable to identify and to remove or at least to reduce T-cell epitopes from a given in principal therapeutically valuable but originally immunogenic peptide, polypeptide or protein. One of these therapeutically valuable molecules is carboxypeptidase G2 (herein abbreviated to CPG2).

CPG2 is a bacterial enzyme (EC number 3.4.17.11) originally isolated from a Pseudomonas species strain RS-16. The enzyme has broad substrate specificity and catalyses the release of C-terminal glutamate residues from a range of different N-acyl groups. The gene encoding the enzyme has been characterized [Minton, N. P. et al (1984) Gene 31: 1-3] and the crystal structure of the protein resolved [Roswell, S. et al (1997) Structure 5: 337-347]. The enzyme has been used previously in an antibody-directed enzyme prodrug therapeutic (ADEPT) strategy for the treatment of cancer and has also been adopted for use in experimental gene-directed enzyme prodrug therapy (GDEPT). In the ADEPT approach, the enzyme molecule is linked to a targeting moiety such as an antibody or fragment of an antibody retaining the antigen binding specificity [Napier, M. P. et al (2000) Clinical Cancer Res. 6: 765-772]. The linkage to the antibody may be via chemical cross-linker or the antibody CPG2 enzyme may by be linked as a fusion protein expressed from a recombinant host organism such as E. coli.

The present invention is therefore concerned with the enzyme CPG2, and the amino acid sequence of the wild-type form of the protein as depicted in single letter code is as follows:

QKRDNVLFQAATDEQPAVIKTLEKLVNIETGTGDAEGIAAAGNFLEAELK
NLGFTVTRSKSAGLVVGDNIVGKIKGRGGKNLLLMSHMDTVYLKGILAKA
PFRVEGDKAYGPGIADDKGGNAVILHTLKLLKEYGVRDYGTITVLFNTDE
EKGSFGSRDLIQEEAKLADYVLSFEPTSAGDEKLSLGTSGIAYVQVNITG
KASHAGAAPELGVNALVEASDLVLRTMNIDDKAKNLRFNWTIAKAGNVSN
IIPASATLNADVRYARNEDFDAAMKTLEERAQQKKLPEADVKVIVTRGRP
AFNAGEGGKKLVDKAVAYYKEAGGTLGVEERTGGGTDAAYAALSGKPVIE
SLGLPGFGYHSDKAEYVDISAIPRRLYMAARLIMDLGAGK

Despite the availability of therapeutic quantities of CPG2 fusion proteins there is a need for enhancement of the in vivo characteristics when administered to the human subject. In this regard, it is highly desired to provide CPG2 with reduced or absent potential to induce an immune response in the human subject. Such proteins would expect to display an increased circulation time within the human subject. The present invention provides for modified forms of CPG2 proteins that are expected to display enhanced properties in vivo.

Others have provided CPG2 molecules [WO88/07378] including modified CPG2 [U.S. Pat. No., 6,004,550] but none of these teachings recognize the importance of T cell epitopes to the immunogenic properties of the protein nor have been conceived to directly influence said properties in a specific and controlled way according to the scheme of the present invention.

It is a particular objective of the present invention to provide modified CPG2 proteins in which the immune characteristic is modified by means of reduced numbers of potential T-cell epitopes.

### SUMMARY AND DESCRIPTION OF THE INVENTION

The present invention provides for modified forms of CPG2, in which the immune characteristic is modified by means of reduced numbers of potential T-cell epitopes. The invention discloses sequences identified within the CPG2 primary sequence that are potential T-cell epitopes by virtue of MHC class II binding potential. This disclosure specifically pertains the mature CPG2 protein of 390 amino acid residues.

The present invention discloses the major regions of the CPG2 primary sequence that are immunogenic in man and thereby provides the critical information required to conduct modification to the sequences to eliminate or reduce the immunogenic effectiveness of these sites.

In one embodiment, synthetic peptides comprising the immunogenic regions can be provided in pharmaceutical composition for the purpose of promoting a tolerogenic response to the whole molecule.

In a further embodiment CPG2 molecules modified within the epitope regions herein disclosed can be used in pharmaceutical compositions.

In summary the invention relates to the following issues:
using a panel of synthetic peptides in a naïve T-cell assay to map the immunogenic region(s) of CPG2;
using a panel of CPG2 protein variants in a naïve T-cell assay to select variants displaying minimal immunogenicity in vitro;
using a panel of synthetic CPG2 peptide variants in a naïve T-cell assay to select peptide sequences displaying minimal immunogenicity in vitro;
using biological assays of T-cell stimulation to select a CPG2 protein variant which exhibits a stimulation index of less than the index recorded for a wild-type CPG2 molecule and preferably less than 2.0 in a naïve T-cell assay;
CPG2 derived peptide sequences originally found to have a stimulation index of greater than 1.8 and preferably greater than 2.0 in a naïve T-cell assay and wherein the peptide is modified to a minimum extent and tested in the naïve T-cell assay and found to have a reduced stimulation index;
CPG2 derived peptide sequences sharing 100% amino acid identity with the wild-type protein sequence and able to evoke a stimulation index of 1.8 or greater and preferably greater than 2.0 in a naïve T-cell assay;
an accordingly specified CPG2 peptide sequence modified to contain less than 100% amino acid identity with the wild-type protein sequence and evoking a stimulation index of less than 2.0 when tested in a T-cell assay;
a CPG2 molecule containing modifications such that when tested in a T-cell assay evokes a reduced stimulation index in comparison to a non modified protein molecule;
a CPG2 molecule in which the immunogenic regions have been mapped using a T-cell assay and then modified such that upon re-testing in a T-cell assay the modified protein evokes a stimulation index smaller than the parental (non-modified) molecule and most preferably less than 2.0;
a modified molecule having the biological activity of CPG2 and being substantially non-immunogenic or less immunogenic than any non-modified molecule having the same biological activity when used in vivo;
an accordingly specified CPG2 molecule, wherein said loss of immunogenicity is achieved by removing one or more T-cell epitopes derived from the originally non-modified molecule;
an accordingly specified CPG2 molecule, wherein said loss of immunogenicity is achieved by reduction in numbers of MHC allotypes able to bind peptides derived from said molecule;
an accordingly specified CPG2 molecule, wherein one T-cell epitope is removed;
an accordingly specified CPG2 molecule, wherein said originally present T-cell epitopes are MHC class II ligands or peptide sequences which show the ability to stimulate or bind T-cells via presentation on class II;
an accordingly specified molecule, wherein said peptide sequences are selected from the group as depicted in Table 1 or Table 2;
an accordingly specified molecule, wherein the molecule contains one or more amino acid substitutions selected from the group as depicted in Table 4 or Table 5;
an accordingly specified molecule, wherein 1-9 amino acid residues, preferably one amino acid residue in any of the originally present T-cell epitopes are altered;
an accordingly specified molecule, wherein the alteration of the amino acid residues is substitution, addition or deletion of originally present amino acid(s) residue(s) by other amino acid residue(s) at specific position(s);
an accordingly specified molecule, wherein, if necessary, additionally further alteration usually by substitution, addition or deletion of specific amino acid(s) is conducted to restore biological activity of the molecule;

an accordingly specified molecule wherein alteration is conducted at one or more residues from any or all of the string of contiguous residues of sequences (A)-(K) as below wherein said sequences are derived from the CPG2 wild-type sequence where using single letter code;

A. = VGKIKGRGGKNLLLMSHMDTVYLKGILAK;
B. = KAYGPGIADDKGGNAVILHTLKLLKEYG;
C. = LFNTDEEKGSFGSRDLIQEEA;
D. = KLADYVLSFEPTSAGDEKLSLGTSG;
E. = VNITGKASHAGAAPELGVNALVEASDL;
F. = KAKNLRFNWTIAKAGNVSNIIPASATLNAD;
G. = ADVKVIVTRGRPAFNAGEGGKKLVDKA;
H. = KKLVDKAVAYYKEAGG;
I. = YKEAGGTLGVEERTGGG;
J. = TDAAYAALSGKPVIESLGLPGFGY
K. = LEKLVNIETGTGDAE;

a peptide molecule comprising at least 9 consecutive residues from any of the sequences (A)-(K) above;
a peptide molecule of above sharing greater than 90% amino acid identity with any of the peptide sequences derived from (A)-(K);
a peptide molecule of above sharing greater than 80% amino acid identity with any of the peptide sequences derived from (A)-(K) above;
a peptide molecule containing a sequence element identical or substantially homologous with one or more of the sequences (A)-(K) above;
peptide sequences as above able to bind MHC class II;
a pharmaceutical composition comprising any of the peptides or modified peptides of above having the activity of binding to MHC class II
a DNA sequence or molecule which codes for any of the specified modified molecules as defined above and below;
a pharmaceutical composition comprising a modified molecule having the biological activity of CPG2;
a pharmaceutical composition as defined above and/or in the claims, optionally together with a pharmaceutically acceptable carrier, diluent or excipient;
a method for manufacturing a modified molecule having the biological activity of CPG2 as defined herein comprising the following steps: (i) determining the amino acid sequence of the polypeptide or part thereof; (ii) identifying one or more potential T-cell epitopes within the amino acid sequence of the protein by any method including determination of the binding of the peptides to MHC molecules using in vitro or in silico techniques or biological assays; (iii) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays; (iv) constructing such sequence variants by recombinant DNA techniques and testing said variants in order to identify one or more variants with desirable properties; and (v) optionally repeating steps (ii)-(iv);
an accordingly specified method, wherein step (iii) is carried out by substitution, addition or deletion of 1-9 amino acid residues in any of the originally present T-cell epitopes;
an accordingly specified method, wherein the alteration is made with reference to an homologous protein sequence and/or in silico modeling techniques;
a peptide sequence consisting of at least 9 consecutive amino acid residues of a 13 mer T-cell epitope peptide as specified above and its use for the manufacture of CPG2 having substantially no or less immunogenicity than any non-modified molecule and having the biological activity of CPG2 when used in vivo;

a CPG2 molecule of the following structure:

X⁰QKRDNVLFQAATDEQPAVIKTLEKLVNIETGTGDAEGIAAAGNFLEAE
LKNLGFTVTRSKSAGLVVGDNIVGKX¹KGRGGKNLX²LX³SHMDTVYX⁴K
GILAKAPFRVEGDKAX⁵GPGX⁶ADDKGGNAX⁷IX⁸HTX⁹X¹⁰X¹¹X¹²KE
YGVRDYGTITVX¹³FNTDEEKGSFGSRDLX¹⁴QEEAKLADYX¹⁵X¹⁶S
X¹⁷EPTSAGDEKX¹⁸SLGTSGIAYVQVNITGKASHAGAAPEX¹⁹GX²⁰NA
X²¹X²²EASDLVLRTMNIDDKAKNX²³RFNX²⁴TX²⁵AKAGX²⁶X²⁷S
X²⁸X²⁹X³⁰PASATX³¹NADVRYARNEDFDAAMKTLEERAQQKKLPEAD
X³²KX³³IVTRGRPAFNAGEGGKX³⁴X³⁵X³⁶DRAX³⁷AX³⁸X³⁹KEAGG
TLGVEERTGGGTDAAYAALSGKPVIESLGLPGFGYHSDKAEYVDISAIPR
RLYMAARLIMDLGAGK

wherein X⁰ is optionally a targeting moiety such as an antibody domain;
X¹ is I, T; X² is L, A; X³ is M, K; X⁴ is L, I; X⁵ is Y, T; X⁶ is I, A; X⁷ is V, A;
X⁸ is L, A; X⁹ is L, T, A; X¹⁰ is K, T; X¹¹ is L, M, A; X¹² is L, T, A; X¹³ is L, G, T;
X¹⁴ is I, T, A; X¹⁵ is V, A; X¹⁶ is L, T, G; X¹⁷ is F, A; X¹⁸ is L, A; X¹⁹ is L, A;
X²⁰ is V, A; X²¹ is L, A; X²² is V, T, A; X²³ is L, A; X²⁴ is W, A, H; X²⁵ is I, A;
X²⁶ is N, T, A; X²⁷ is V, T; X²⁸ is N, T, A; X²⁹ is I, T, A; X³⁰ is I, T, A;
X³¹ is L, T, A, I; X³² is V, A; X³³ is V, A; X³⁴ is K, T; X³⁵ is L, A; X³⁶ is V, A;
X³⁷ is V, S, T, A; X³⁸ is Y, T, A; X³⁹ is Y, S, T, A;
   and whereby simultaneously X1=I, X2=L, X3=M, X4=L, X5=Y, X6=I, X7=V, X8 =L, X9=L, X10=K, X11=L, X12=L, X13=L, X14=I, X15=V, X16=L, X17=F, X18=L, X19=L, X20=V, X21=L, X22=V, X23=L, X24=W, X25=I, X26=N, X27=V, X28=N, X29=I, X30=I, X31=L, X32=V, X33=V, X34=K, X35=L, X36=V, X37=V, X38=Y and X39=Y are excluded.

The term “T-cell epitope” means according to the understanding of this invention an amino acid sequence which is able to bind MHC class II, able to stimulate T-cells and/or also to bind (without necessarily measurably activating) T-cells in complex with MHC class II.

The term “peptide” as used herein and in the appended claims, is a compound that includes two or more amino acids. The amino acids are linked together by a peptide bond (defined herein below). There are 20 different naturally occurring amino acids involved in the biological production of peptides, and any number of them may be linked in any order to form a peptide chain or ring. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Some peptides contain only a few amino acid units. Short peptides, e.g., having less than ten amino acid units, are sometimes referred to as “oligopeptides”. Other peptides contain a large number of amino acid residues, e.g. up to 100 or more, and are referred to as “polypeptides”. By convention, a “polypeptide” may be considered as any peptide chain containing three or more amino acids, whereas a “oligopeptide” is usually considered as a particular type of “short” polypeptide. Thus, as used herein, it is understood that any reference to a “polypeptide” also includes an oligopeptide. Further, any reference to a “peptide” includes polypeptides, oligopeptides, and proteins. Each different arrangement of amino acids forms different polypeptides or proteins. The number of polypeptides—and hence the number of different proteins—that can be formed is practically unlimited.

“Alpha carbon (Cα)” is the carbon atom of the carbon-hydrogen (CH) component that is in the peptide chain. A “side chain” is a pendant group to Cα that can comprise a simple or complex group or moiety, having physical dimensions that can vary significantly compared to the dimensions of the peptide.

The invention may be applied to any CPG2 species of molecule with substantially the same primary amino acid sequences as those disclosed herein and would include therefore CPG2 molecules derived by genetic engineering means or other processes and may not contain 390 amino acid residues. CPG2 proteins such as identified from other sources including different strains of Pseudomonas and other organisms have in common many of the peptide sequences of the present disclosure and have in common many peptide sequences with substantially the same sequence as those of the disclosed listing. Such protein sequences equally therefore fall under the scope of the present invention.

The invention is conceived to overcome the practical reality that soluble proteins introduced with therapeutic intent in man trigger an immune response resulting in development of host antibodies that bind to the soluble protein. The present invention seeks to address this by providing CPG2 □proteins with altered propensity to elicit an immune response on administration to the human host. According to the methods described herein, the inventors have discovered the regions of the CPG2 molecule comprising the critical T-cell epitopes driving the immune responses to this protein.

The general method of the present invention leading to the modified CPG2 comprises the following steps:
(a) determining the amino acid sequence of the polypeptide or part thereof;
(b) identifying one or more potential T-cell epitopes within the amino acid sequence of the protein by any method including determination of the binding of the peptides to MHC molecules using in vitro or in silico techniques or biological assays;
(c) designing new sequence variants with one or more amino acids within the identified potential T-cell epitopes modified in such a way to substantially reduce or eliminate the activity of the T-cell epitope as determined by the binding of the peptides to MHC molecules using in vitro or in silico techniques or biological assays. Such sequence variants are created in such a way to avoid creation of new potential T-cell epitopes by the sequence variations unless such new potential T-cell epitopes are, in turn, modified in such a way to substantially reduce or eliminate the activity of the T-cell epitope; and
(d) constructing such sequence variants by recombinant DNA techniques and testing said variants in order to identify one or more variants with desirable properties according to well known recombinant techniques.

The identification of potential T-cell epitopes according to step (b) can be carried out according to methods described previously in the art. Suitable methods are disclosed in WO 98/59244; WO 98/52976; WO 00/34317 and may be used to identify binding propensity of CPG2-derived peptides to an MHC class II molecule.

Another very efficacious method for identifying T-cell epitopes by calculation is described in the Example 1 which is a preferred embodiment according to this invention.

The results of an analysis according to step (b) of the above scheme and pertaining to the CPG2 protein sequence is presented in Table 1.

TABLE 1

Peptide sequences in CPG2 with potential human MHC
class II binding activity.

DNVLFQAATDEQP, NVLFQAATDEQPA, VLFQAATDEQPAV,
PAVIKTLEKLVNI, AVIKTLEKLVNIE, KTLEKLVNIETGT,
EKLVNIETGTGDA, KLVNIETGTGDAE, VNIETGTGDAEGI,
EGIAAAGNFLEAE, GNFLEAELKNLGF, NFLEAELKNLGFT,
AELKNLGFTVTRS, KNLGFTVTRSKSA, LGFTVTRSKSAGL,
FTVTRSKSAGLVV, AGLVVGDNIVGKI, GLVVGDNIVGKIK,
LVVGDNIVGKIKG, DNIVGKIKGRGGK, NIVGKIKGRGGKN,
GKIKGRGGKNLLL, KNLLLMSHMDTVY, NLLLMSHMDTVYL,
LLLMSHMDTVYLK, LLMSHMDTVYLKG, SHMDTVYLKGILA,
DTVYLKGILAKAP, TVYLKGILAKAPF, VYLKGILAKAPFR,
KGILAKAPFRVEG, GILAKAPFRVEGD, APFRVEGDKAYGP,
FRVEGDKAYGPGI, KAYGPGIADDKGG, PGIADDKGGNAVI,
NAVILHTLKLLKE, AVILHTLKLLKEY, VILHTLKLLKEYG,
HTLKLLKEYGVRD, LKLLKEYGVRDYG, KLLKEYGVRDYGT,
KEYGVRDYGTITV, YGVRDYGTITVLF, RDYGTITVLFNTD,
GTITVLFNTDEEK, ITVLFNTDEEKGS, TVLFNTDEEKGSF,
VLFNTDEEKGSFG, GSFGSRDLIQEEA, RDLIQEEAKLADY,
DLIQEEAKLADYV, AKLADYVLSFEPT, ADYVLSFEPTSAG,
DYVLSFEPTSAGD, YVLSFEPTSAGDE, LSFEPTSAGDEKL,
EKLSLGTSGIAYV, LSLGTSGIAYVQV, SGIAYVQVNITGK,
IAYVQVNITGKAS, AYVQVNITGKASH, VQVNITGKASHAG,
VNITGKASHAGAA, PELGVNALVEASD, LGVNALVEASDLV,
NALVEASDLVLRT, ALVEASDLVLRTM, SDLVLRTMNIDDK,
DLVLRTMNIDDKA, LVLRTMNIDDKAK, RTMNIDDKAKNLR,
MNIDDKAKNLRFN, KNLRFNWTIAKAG, LRFNWTIAKAGNV,
FNWTIAKAGNVSN, WTIAKAGNVSNII, GNVSNIIPASATL,
SNIIPASATLNAD, NIIPASATLNADV, ATLNADVRYARNE,
ADVRYARNEDFDA, VRYARNEDFDAAM, EDFDAAMKTLEER,
AAMKTLEERAQQK, KTLEERAQQKKLP, KKLPEADVKVIVT,
ADVKVIVTRGRPA, VKVIVTRGRPAFN, KVIVTRGRPAFNA,
VIVTRGRPAFNAG, PAFNAGEGGKKLV, KKLVDKAVAYYKE,
KLVDKAVAYYKEA, KAVAYYKEAGGTL, VAYYKEAGGTLGV,
AYYKEAGGTLGVE, GTLGVEERTGGGT, LGVEERTGGGTDA,
AAYAALSGKPVIE, AALSGKPVIESLG, KPVIESLGLPGFG,
PVIESLGLPGFGY, ESLGLPGFGYHSD, LGLPGFGYHSDKA,
PGFGYHSDKAEYV, FGYHSDKAEYVDI, AEYVDISAIPRRL,
EYVDISAIPRRLY, VDISAIPRRLYMA, SAIPRRLYMAARL,
RRLYMAARLIMDL, RLYMAARLIMDLG, LYMAARLIMDLGA

A further important technical approach for the detection of T-cell epitopes is via biological T-cell proliferation assay. For the detection of T-cell epitopes within the CPG2 molecule a particularly effective method is to test overlapping peptides derived from the CPG2 sequence so as to test the entire CPG2 sequence, or alternatively to test a sub-set of CPG2 peptides such as all or some of those listed in Table 1. The synthetic peptides are tested for their ability to evoke a proliferative response in human T-cells cultured in vitro. This type of approach can be conducted using naïve human T-cells taken from healthy donors. The inventors have established that in the operation of such an assay, a stimulation index equal to or greater than 2.0 is a useful measure of induced proliferation.

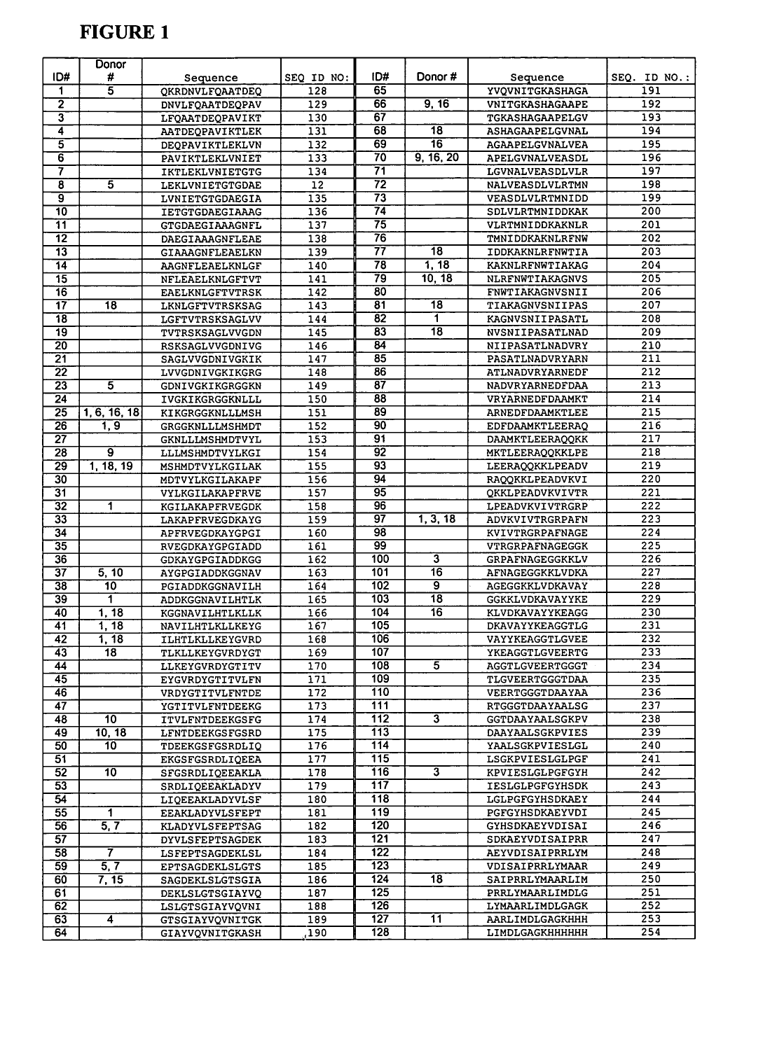

The stimulation index is conventionally derived by division of the proliferation score measured (e.g. counts per minute if using ³H-thymidine incorporation) to the test (poly) peptide by the proliferation score measured in cells not contacted with a test (poly) peptide. A suitable method of this type is detailed in Example 2. Results from this assay are presented in Table 2 and FIG. 1 where are listed CPG2 derived peptide sequences shown by the method of example 2 to evoke a proliferative response in human T-cells.

TABLE 2

CPG2 peptide sequences able to stimulate human
T-cells in vitro.

| | | |
|---|---|---|
| Peptide | | |
| ID # | Peptide Sequence | Residue # |

| | | |
|---|---|---|
| 1 | QKRDNVLFQAATDEQ | 1 |
| 8 | LEKLVNIETGTGDAE | 22 |
| 17 | LKNLGFTVTRSKSAG | 49 |
| 23 | GDNIVGKIKGRGGKN | 67 |
| 25 | KIKGRGGKNLLLMSH | 73 |
| 26 | GRGGKNLLLMSHMDT | 76 |
| 28 | LLLMSHMDTVYLKGI | 82 |
| 29 | MSHMDTVYLKGILAK | 85 |
| 32 | KGILAKAPFRVEGDK | 94 |
| 37 | AYGPGIADDKGGNAV | 109 |
| 38 | PGIADDKGGNAVILH | 112 |
| 39 | ADDKGGNAVILHTLK | 115 |
| 40 | KGGNAVILHTLKLLK | 118 |
| 41 | NAVILHTLKLLKEYG | 121 |
| 42 | ILHTLKLLKEYGVRD | 124 |
| 43 | TLKLLKEYGVRDYGT | 127 |
| 48 | ITVLFNTDEEKGSFG | 142 |
| 49 | LFNTDEEKGSFGSRD | 145 |
| 50 | TDEEKGSFGSRDLIQ | 148 |
| 52 | SFGSRDLIQEEAKLA | 154 |
| 55 | EEAKLADYVLSFEPT | 163 |
| 56 | KLADYVLSFEPTSAG | 166 |
| 58 | LSFEPTSAGDEKLSL | 172 |
| 59 | EPTSAGDEKLSLGTS | 175 |
| 60 | SAGDEKLSLGTSGIA | 178 |
| 63 | GTSGIAYVQVNITGK | 187 |
| 66 | VNITGKASHAGAAPE | 196 |
| 68 | ASHAGAAPELGVNAL | 202 |
| 69 | AGAAPELGVNALVEA | 205 |
| 70 | APELGVNALVEASDL | 208 |
| 77 | IDDKAKNLRFNWTIA | 229 |
| 78 | KAKNLRFNWTIAKAG | 232 |
| 79 | NLRFNWTIAKAGNVS | 235 |
| 81 | TIAKAGNVSNIIPAS | 241 |
| 82 | KAGNVSNIIPASATL | 244 |
| 83 | NVSNIIPASATLNAD | 247 |
| 97 | ADVKVIVTRGRPAFN | 289 |
| 100 | GRPAFNAGEGGKKLV | 298 |
| 101 | AFNAGEGGKKLVDKA | 301 |
| 102 | AGEGGKKLVDKAVAY | 304 |
| 103 | GGKKLVDKAVAYYKE | 307 |
| 104 | KLVDKAVAYYKEAGG | 310 |
| 108 | AGGTLGVEERTGGGT | 322 |
| 112 | GGTDAAYAALSGKPV | 334 |
| 116 | KPVIESLGLPGFGYH | 346 |
| 124 | SAIPRRLYMAARLIM | 370 |
| 127 | AARLIMDLGAGK | 379 |

Where multiple potential epitopes are identified and in particular where a number of peptide sequences are found to be able to stimulate T-cells in a biological assay, cognizance may also be made of the structural features of the protein in relation to its propensity to evoke an immune response via the MHC class II presentation pathway. For example where the crystal structure of the protein of interest is known the crystallographic B-factor score may be analyzed for evidence of structural disorder within the protein, a parameter suggested to correlate with the proximity to the biologically relevant immunodominant peptide epitopes [Dai G. et al (2001) J. Biological Chem. 276: 41913-41920]. Such an analysis when conducted on the CPG2 crystal structure [PDB ID: 1CG2, Rowsell, S. et al (997), Structure 5: 337] suggests a high likelihood for multiple immunodominant epitopes with at least 11 discrete zones mapping to the medial position of areas with above average B-factor scores in at least three of the four individual chains in the CPG2 modeled crystal structure. Of the these 11 areas, 9 mapped to the N-terminal boundary of peptides shown to evoke a proliferative response in the naïve T-cell assay of example 2.

This data taken together with the data for the numbers of naïve donors responding to particular peptides enables a predicted ranking of the most immunodominant regions of the molecule. It is however recognized that in practice, each of these regions are considered immunogenic in man and therefore require modification under the scheme of the invention. Accordingly, with reference to the above defined sequence strings (A)-(K), sequences may be ranked in the order (A), {(B), (E)}, {(D), (G), (F), (H)}, {(I), (J), (K)}; where (A) is considered the most immunogenic sequence within the molecule. Equal ranking is ascribed to those sequences in brackets. These regions have been listed in Table 3 as the major epitope regions A-H and the lesser epitope regions I and J. This distinction has been drawn based on the frequency of responding donors in the naïve T-cell assay, whereby for the so called “lesser epitope” regions, T-cells from one donor of the panel where shown to respond to the synthetic peptide representative of this region of sequence. By contrast, peptides mapping to the major epitope regions were for the most part stimulatory to T-cells from multiple donors. The donor panel used was representative of a wide repertoire of different MHC class II allotypes.

TABLE 3
CPG2 Epitope regions

| | |
|---|---|
| Sequence | Residue # |

| | | |
|---|---|---|
| Major | | |
| Epitope | | |
| Region | | |
| A | VGKIKGRGGKNLLLMSHMDTVYLKGILAK | 71-99 |
| B | KAYGPGIADDKGGNAVILHTLKLLKEYG | 108-135 |
| C | LFNTDEEKGSFGSRDLIQEEA | 145-165 |
| D | KLADYVLSFEPTSAGDEKLSLGTSG | 166-190 |
| E | VNITGKASHAGAAPELGVNALVEASDL | 196-222 |
| F | KAKNLRFNWTIAKAGNVSNIIPASATLNAD | 232-261 |
| G | ADVKVIVTRGRPAFNAGEGGKKLVDKA | 289-315 |
| H | KKLVDKAVAYYKEAGG | 309-324 |
| Lesser | | |
| Regions | | |
| I | YKEAGGTLGVEERTGGG | 319-235 |
| J | TDAAYAALSGKPVIESLGLPGFGY | 236-259 |
| K | LEKLVNIETGTGDAE | 25-39 |

In practice a number of variant CPG2 proteins will be produced and tested for the desired immune and functional characteristic. The variant proteins will most preferably be produced by the widely known methods of recombinant DNA technology although other procedures including chemical synthesis of CPG2 fragments may be contemplated. Suitable methods for the construction and expression of CPG2 proteins including a modified CPG2 protein are provided in the Examples 3-5.

The invention relates to CPG2 analogues in which substitutions of at least one amino acid residue have been made at positions resulting in a substantial reduction in activity of or elimination of one or more potential T-cell epitopes from the protein. It is most preferred to provide CPG2 molecules in which amino acid modification (e.g. a substitution) is conducted within the most immunogenic regions of the parent molecule. The major preferred embodiments of the present invention comprise CPG2 molecules for which any of the MHC class II ligands are altered such as to eliminate binding or otherwise reduce the numbers of MHC allotypes to which the peptide can bind. The inventors have discovered and herein disclose, the immunogenic regions of the CPG2 molecule in man. It is understood that under certain circumstances additional regions of sequence to those disclosed herein can become immunogenic epitopes, for example in the event of infection with a pathogen expressing a protein or peptide with a similar sequence to that of the present case. In any event, it will be critical for the sequence element to act as an MHC class II ligand and in principle therefore any of the sequences disclosed in Table 1 can be considered immunogenic epitopes under the scope of the present invention.

For the elimination of T-cell epitopes, amino acid substitutions are preferably made at appropriate points within the peptide sequence predicted to achieve substantial reduction or elimination of the activity of the T-cell epitope. In practice an appropriate point will preferably equate to an amino acid residue binding within one of the pockets provided within the MHC class II binding groove.

It is most preferred to alter binding within the first pocket of the cleft at the so-called P1 or P1 anchor position of the peptide. The quality of binding interaction between the P1 anchor residue of the peptide and the first pocket of the MHC class II binding groove is recognized as being a major determinant of overall binding affinity for the whole peptide. An appropriate substitution at this position of the peptide will be for a residue less readily accommodated within the pocket, for example, substitution to a more hydrophilic residue. Amino acid residues in the peptide at positions equating to binding within other pocket regions within the MHC binding cleft are also considered and fall under the scope of the present.

It is understood that single amino acid substitutions within a given potential T-cell epitope are the most preferred route by which the epitope may be eliminated. Combinations of substitution within a single epitope may be contemplated and for example can be particularly appropriate where individually defined epitopes are in overlap with each other. Moreover, amino acid substitutions either singly within a given epitope or in combination within a single epitope may be made at positions not equating to the “pocket residues” with respect to the MHC class II binding groove, but at any point within the peptide sequence. Substitutions may be made with reference to an homologues structure or structural method produced using in silico techniques known in the art and may be based on known structural features of the molecule according to this invention. All such substitutions fall within the scope of the present invention.

Amino acid substitutions other than within the peptides identified above may be contemplated particularly when made in combination with substitution(s) made within a listed peptide. For example a change may be contemplated to restore structure or biological activity of the variant molecule. Such compensatory changes and changes to include deletion or addition of particular amino acid residues from the CPG2 polypeptide resulting in a variant with desired activity and in combination with changes in any of the disclosed peptides fall under the scope of the present.

A particularly preferred set of mutations within the CPG2 molecule contemplated under the scheme of the present are listed in Table 4. Such substitutions are selected using the computational methods of Example 1. Each of the substitutions map to the epitope regions defined herein above and listed also as Table 3.

TABLE 4
Substitutions in CPG2

| | | |
|---|---|---|
| Epitope | | |
| Region | CPG2 Peptide | Substitution within CPG2 peptide |

| | | | | | | | |
|---|---|---|---|---|---|---|---|
| A | DNIVGKIKGRGGK | WT Residue | I | G | | | |
| | | Position | 3 | 11 | | | |
| | | | C, G, P, D, | | | | |
| | | Substitution | E, H, K, N, | I, P, T | | | |
| | | | Q, R, S, T | | | | |
| A | NIVGKIKGRGGKN | WT Residue | V | G | | | |
| | | Position | 3 | 11 | | | |
| | | | A, C, D, E, | | | | |
| | | | G, H, K, N, | | | | |
| | | Substitution | | T | | | |
| | | | P, Q, R, S, | | | | |
| | | | T | | | | |
| A | GKIKGRGGKNLLL | WT Residue | I | G | L | | |
| | | Position | 3 | 8 | 11 | | |
| | | | | | C, D, E, G, | | |
| | | | A, C, D, E, | | | | |
| | | | | | H, I, K, M, | | |
| | | Substitution | H, K, N, P, | H, P | | | |
| | | | | | N, P, Q, R, | | |
| | | | Q, R, S, T | | | | |
| | | | | | S, T, V, W | | |
| A | NLLLMSHMDTVYL | WT Residue | L | S | M | D | V |
| | | Position | 3 | 6 | 8 | 9 | 11 |
| | | | A, C, D, E, | | | | |
| | | | F, G, H, I, | | D, E, H, K, | | |
| | | Substitution | K, M, N, P, | P | N, P, Q, | P | T |
| | | | Q, R, S, T, | R,, S, T | | | |
| | | | V, W, Y | | | | |
| A | LLMSHMDTVYLKG | WT Residue | M | V | L | | |
| | | Position | 3 | 9 | 11 | | |
| | | | A, C, D, E, | | | | |
| | | | G, H, K, N, | | | | |
| | | Substitution | | P | I, P, T, W | P | |
| | | | P, Q, R, S, | | | | |
| | | | T | | | | |
| A | DTVYLKGILAKAP | WT Residue | V | I | L | | |
| | | Position | 3 | 8 | 9 | | |
| | | | A, C, D, E, | | | | |
| | | | G, H, K, N, | | | | |
| | | Substitution | | P, W, Y | P | | |
| | | | P, Q, R, S, | | | | |
| | | | T | | | | |
| A | TVYLKGILAKAPF | WT Residue | Y | | | | |
| | | Position | 3 | | | | |
| | | | A, C, G, M, | | | | |
| | | Substitution | | | | | |
| | | | P | | | | |
| B | KAYGPGIADDKGG | WT Residue | Y | G | A | D | K |
| | | Position | 3 | 6 | 8 | 9 | 11 |
| | | | A, C, D, E, | | | | |
| | | | G, H, K, M, | | | | |
| | | Substitution | | P | F, L, W, Y | P | I, P, W |
| | | | N, P, Q, R, | | | | |
| | | | S, T, W | | | | |
| B | PGIADDKGGNAVI | WT Residue | I | D | G | G | A |
| | | Position | 3 | 6 | 8 | 9 | 11 |
| | | | A, C, D, E, | | | | |
| | | | | | | | D, E, H, K, |
| | | | G, H, K, M, | | | D, H, K, N, | |
| | | Substitution | | P | H, P, Q, S | | N, P, Q, R, |
| | | | N, P, Q, R, | | | P, Q, R, S, | |
| | | | | | | | S, T |
| | | | S, T, W | | | T | |
| B | NAVILHTLKLLKE | WT Residue | V | H | K | | |
| | | Position | 3 | 6 | 9 | | |
| | | | A, C, D, E, | | | | |
| | | | G, H, K, M, | | | | |
| | | Substitution | | P | P, T | | |
| | | | N, P, Q, R, | | | | |
| | | | S, T, W | | | | |
| B | VILHTLKLLKEYG | WT Residue | L | | | | |
| | | Position | 3 | | | | |
| | | | A, C, D, E, | | | | |
| | | | G, H, K, N, | | | | |
| | | Substitution | | | | | |
| | | | P, Q, R, S, | | | | |
| | | | T | | | | |
| B | HTLKLLKEYGVRD | WT Residue | L | | | | |
| | | Position | 3 | | | | |
| | | | A, C, D, E, | | | | |
| | | | G, H, K, N, | | | | |
| | | Substitution | | | | | |
| | | | P, Q, R, S, | | | | |
| | | | T | | | | |
| B | LKLLKEYGVRDYG | WT Residue | L | V | | | |
| | | Position | 3 | 9 | | | |
| | | | A, C, D, E, | | | | |
| | | | G, H, K, M, | | | | |
| | | Substitution | | P | | | |
| | | | N, P, Q, R, | | | | |
| | | | S, T, W, Y | | | | |
| B | KLLKEYGVRDYGT | WT Residue | L | | | | |
| | | Position | 3 | | | | |
| | | | A, C, D, E, | | | | |
| | | | G, H, K, N, | | | | |
| | | Substitution | | | | | |
| | | | P, Q, R, S, | | | | |
| | | | T | | | | |
| C | TVLFNTDEEKGSF | WT Residue | L | G | | | |
| | | Position | 3 | 11 | | | |
| | | | A, C, D, E, | | | | |
| | | | | D, E, H, K, | | | |
| | | | G, H, K, M, | | | | |
| | | Substitution | | N, P, Q, R, | | | |
| | | | N, P, Q, R, | | | | |
| | | | | S, T, W | | | |
| | | | S, T, W | | | | |
| C | RDLIQEEAKLADY | WT Residue | L | | | | |
| | | Position | 3 | | | | |
| | | Substitution | P | | | | |
| C | DLIQEEAKLADYV | WT Residue | I | L | D | | |
| | | Position | 3 | 9 | 11 | | |
| | | | A, C, D, E, | | | | |
| | | | | C, D, E, G, | | | |
| | | | G, H, K, N, | | | | |
| | | Substitution | | H, K, N, P, | T | | |
| | | | P, Q, R, S, | | | | |
| | | | | Q, R, S, T | | | |
| | | | T | | | | |
| D | DYVLSFEPTSAGD | WT Residue | V | F | A | | |
| | | Position | 3 | 6 | 11 | | |
| | | | A, C, G, P | | | | |
| | | Substitution | | P | P, W | | |
| | | | W, Y | | | | |
| D | YVLSFEPTSAGDE | WT Residue | L | | | | |
| | | Position | 3 | | | | |
| | | Substitution | T, Q, R | | | | |
| D | LSFEPTSAGDEKL | WT Residue | F | T | A | G | E |
| | | Position | 3 | 6 | 8 | 9 | 11 |
| | | | | | | E, N, P, Q, | |
| | | Substitution | A, C, G, P | P | P, W | | P, T, W |
| | | | | | | R, S, T | |
| D | EKLSLGTSGIAYV | WT Residue | L | | | | |
| | | Position | 3 | | | | |
| | | | A, C, G, P, | | | | |
| | | Substitution | | | | | |
| | | | W | | | | |
| E | PELGVNALVEASD | WT Residue | L | N | V | A | |
| | | Position | 3 | 6 | 9 | 11 | |
| | | | A, C, D, E, | | | | |
| | | | F, G, H, I, | | | | |
| | | | | | E, H, K, N, | D, E, H, N, | |
| | | Substitution | K, M, N, P, | P | | | |
| | | | | | P, Q, R, S, | P, Q, S, T | |
| | | | Q, R, S, T, | | | | |
| | | | | | T | | |
| | | | V, W, Y | | | | |
| E | LGVNALVEASDLV | WT Residue | V | L | A | D | |
| | | Position | 3 | 6 | 9 | 11 | |
| | | | A, C, D, E, | A, C, D, E, | | | |
| | | | | | D, E, H, K, | | |
| | | | G, H, K, N, | G, H, I, K, | | | |
| | | Substitution | | | N, P, Q, R, | P | |
| | | | P, Q, R, S, | M, N, P, Q, | | | |
| | | | | | S, T | | |
| | | | T | R, S, T, V, | | | |
| | | | | W, Y | | | |
| E | NALVEASDLVLRT | WT Residue | L | | | | |
| | | Position | 3 | | | | |
| | | | A, C, D, E, | | | | |
| | | | G, H, K, N, | | | | |
| | | Substitution | | | | | |
| | | | P, Q, R, S, | | | | |
| | | | T | | | | |
| F | KNLRFNWTIAKAG | WT Residue | L | I | K | | |
| | | Position | 3 | 9 | 11 | | |
| | | | A, C, G, M, | | | | |
| | | Substitution | | P | T | | |
| | | | P, W | | | | |
| F | FNWTIAKAGNVSN | WT Residue | W | A | A | G | |
| | | Position | 3 | 6 | 8 | 9 | |
| | | | | C, D, H, K, | | | |
| | | | | | D, E, H, K, | | |
| | | Substitution | A, C, G, P | N, P, Q, R, | | P | |
| | | | | | N, P, Q, W, | | |
| | | | | S, T | | | |
| | | | | | Y | | |
| F | WTIAKAGNVSNII | WT Residue | I | A | N | V | N |
| | | Position | 3 | 6 | 8 | 9 | 11 |
| | | | A, C, D, E, | | | | |
| | | | G, H, K, M, | D, E, H, K, | | | |
| | | Substitution | | | F, L, P, W | G, P | P, T, W |
| | | | N, P, Q, R, | N, P, Q | | | |
| | | | S, T, W | | | | |
| F | GNVSNIIPASATL | WT Residue | V | I | A | A | |
| | | Position | 3 | 6 | 9 | 11 | |
| | | | A, C, D, E, | | | | |
| | | | G, H, K, N, | | | | |
| | | Substitution | | P | P | P | |
| | | | P, Q, R, S, | | | | |
| | | | T, W | | | | |
| F | SNIIPASATLNAD | WT Residue | I | A | | | |
| | | Position | 3 | 8 | | | |
| | | | A, C, D, E, | | | | |
| | | | G, H, K, N, | | | | |
| | | Substitution | | H | | | |
| | | | P, Q, R, S, | | | | |
| | | | T | | | | |
| F | NIIPASATLNADV | WT Residue | I | L | A | | |
| | | Position | 3 | 9 | 11 | | |
| | | | A, C, D, E, | | | | |
| | | | | D, E, H, K, | | | |
| | | | G, H, K, N, | | P, I, Q, S, | | |
| | | Substitution | | N, P, Q, R, | | | |
| | | | P, Q, R, S, | | T, V, W | | |
| | | | | S, T | | | |
| | | | T | | | | |
| G | KKLPEADVKVIVT | WT Residue | L | | | | |
| | | Position | 3 | | | | |
| | | | A, C, D, E, | | | | |
| | | | G, H, K, N, | | | | |
| | | Substitution | | | | | |
| | | | P, Q, R, S, | | | | |
| | | | T | | | | |
| G | PAFNAGEGGKKLV | WT Residue | F | G | G | G | K |
| | | Position | 3 | 6 | 8 | 9 | 11 |
| | | | | D, E, H, K, | | | |
| | | | A, C, G, M, | | | | |
| | | Substitution | | P | H, P, S | N, P, Q, R, | P, T, W |
| | | | P, W | | | | |
| | | | | | | S, T | |
| H | KKLVDKAVAYYKE | WT Residue | L | | | | |
| | | Position | 3 | | | | |
| | | | A, C, D, E, | | | | |
| | | | G, H, K, N, | | | | |
| | | Substitution | | | | | |
| | | | P, Q, R, S, | | | | |
| | | | T | | | | |
| H | KLVDKAVAYYKEA | WT Residue | V | | | | |
| | | Position | 3 | | | | |
| | | | A, C, D, E, | | | | |
| | | | G, H, K, N, | | | | |
| | | Substitution | | | | | |
| | | | P, Q, R, S, | | | | |
| | | | T | | | | |
| H | KAVAYYKEAGGTL | WT Residue | V | Y | E | A | G |
| | | Position | 3 | 6 | 8 | 9 | 11 |
| | | | A, C, D, E, | | | | |
| | | | G, H, K, N, | | | | I, P, T, S, |
| | | Substitution | | P | H | P, T | |
| | | | P, Q, R, S, | | | | W, Y |
| | | | T | | | | |
| H | VAYYKEAGGTLGV | WT Residue | Y | G | G | L | |
| | | Position | 3 | 8 | 9 | 11 | |
| | | | A, C, D, E, | | | A, C, D, E, | |
| | | | G, H, K, N, | D, E, H, N, | | F, G, H, I, | |
| | | Substitution | | | H, P | | |
| | | | P, Q, R, S, | P, Q | | K, M, N, P, | |
| | | | T | | | Q, R, S, T, | |
| | | | | | | V, W | |
| H | AYYKEAGGTLGVE | WT Residue | Y | A | G | G | |
| | | Position | 3 | 6 | 8 | 11 | |
| | | | A, C, D, E, | | | | |
| | | | | D, E, H, K, | D, E, F, H, | | |
| | | | G, H, K, M, | | | | |
| | | Substitution | | N, P, Q, R, | L, N, P, Q, | P, T | |
| | | | N, P, Q, R, | | | | |
| | | | | S, T | W, Y | | |
| | | | S, T | | | | |
| I | GTLGVEERTGGGT | WT Residue | L | E | R | T | G |
| | | Position | 3 | 6 | 8 | 9 | 11 |
| | | | A, C, D, E, | | | | |
| | | | F, G, H, I, | | | | D, E, H, K, |
| | | Substitution | K, M, N, P, | P | H, P | P | N, P, Q, R, |
| | | | Q, R, S, T, | | | | S, T |
| | | | V, W, Y | | | | |
| J | AALSGKPVIESLG | WT Residue | L | V | I | S | |
| | | Position | 3 | 8 | 9 | 11 | |
| | | | A, C, D, E, | | | | |
| | | | | D, E, F, H, | D, E, H, K, | | |
| | | | G, H, K, M, | | | | |
| | | Substitution | | K, N, P, Q, | N, P, Q, R, | T | |
| | | | N, P, Q, R, | | | | |
| | | | | S, T, W, Y | S, T | | |
| | | | S, T, W, Y | | | | |
| J | KPVIESLGLPGFG | WT Residue | V | L | G | | |
| | | Position | 3 | 9 | 11 | | |
| | | | D, E, H, K, | | | | |
| | | Substitution | N, P, Q, S, | P | T | | |
| | | | T | | | | |

It is recognized that other known methods could be applied and which may result in definition of particular alternative substitutions. In all events, an especially desired substitution, will be one which can satisfy the parallel objectives of retaining functional activity in the molecule and yet disrupt the ability of the peptide sequence within the locale to act as a ligand for one or more human MHC class II molecules and or cease to stimulate a cognate T-cell receptor. One known and applicable scheme could involve random mutagenesis of the epitope regions disclosed herein and selection of enzymatically functional variants. The selected variant may then be passed to an independent second screen for immunological analysis. A convenient immunological screen for example would be a T-cell proliferation assay using synthetic peptides of the variant sequence and human T-cells or T-cell lines cultured in vitro.

A further method may exploit molecular modeling techniques to select in silico substitutions compatible with the parallel dual objectives outlined above. The structural model of the CPG2 molecule may be examined using any suitable software package and highly desired substitutions may be selected. Examples of such especially preferred substitutions are provided in Table 5; the substitutions are considered broadly accommodated within the CPG2 structure and variant CPG2 molecules containing any of these listed substitutions are to be considered as preferred embodiments of the present invention.

TABLE 5
Preferred substitutions in CPG2

| | Epitope | Wt Residue & # | Substitution |
|---|---|---|---|
| 1 | A1 | I 74 | T |
| 2 | A2 | L 83 | A or G |
| 3 | A3 | M 85 | K |
| 4 | A4 | L 93 | I |
| 5 | B1 | Y 110 | T |
| 6 | B2 | I 114 | A or G |
| 7 | B3 | V 123 | A or G |
| 8 | B4 | L 125 | A or G |
| 9 | B5 | L 128 | T, A |
| 10 | B6 | K 129 | T |
| 11 | B7 | L 130 | M, A |
| 12 | B8 | L 131 | T, A |
| 13 | C1 | L 145 | G or T |
| 14 | C2 | I 161 | T, A |
| 15 | D1 | V 171 | A/G |
| 16 | D2 | L 172 | T or G or R |
| 17 | D3 | F 174 | A or G |
| 18 | D4 | L 184 | A or G |
| 19 | E1 | L 211 | A or G |
| 20 | E2 | V 213 | A or (D, E, G, K, N, Q, S, T,) |
| 21 | E3 | L 216 | A |
| 22 | E4 | V 217 | T, A |
| 23 | F1 | L 236 | A or G |
| 24 | F2 | W 240 | A/G (D, K, N, Q, S, T) |
| 25 | F3 | I 242 | A or T |
| 26 | F4 | N 247 | T, A |
| 27 | F5 | V 248 | T or (H, N, Q, S) |
| 28 | F6 | N 250 | T, A |
| 29 | F7 | I 251 | T, A |
| 30 | F8 | I 252 | T, A |
| 31 | F9 | L 258 | T, A, I |
| 32 | G1 | V 291 | A or G |
| 33 | G2 | V 293 | A or G |
| 34 | G3 | K 310 | T |
| 35 | H1 | L 311 | A or G |
| 36 | H2 | V 312 | A or G |
| 37 | H3 | V 316 | S, T, A |
| 38 | H4 | Y 318 | T, A |
| 39 | H5 | Y 319 | S, T, A |

Accordingly, a particularly preferred modified CPG2 molecule is given by the structure below:

X⁰QKRDNVLFQAATDEQPAVIKTLEKLVNIETGTGDAEGIAAAGNFLEAE
LKNLGFTVTRSKSAGLVVGDNIVGKX¹KGRGGKNLX²LX³SHMDTVYX⁴K
GILAKAPFRVEGDKAX⁵GPGX⁶ADDKGGNAX⁷IX⁸HTX⁹X¹⁰X¹¹X¹²KE
YGVRDYGTITVX¹³FNTDEEKGSFGSRDLX¹⁴QEEAKLADYX¹⁵X¹⁶S
X¹⁷EPTSAGDEKX¹⁸SLGTSGIAYVQVNITGKASHAGAAPEX¹⁹GX²⁰NA
X²¹X²²EASDLVLRTMNIDDKAKNX²³RFNX²⁴TX²⁵AKAGX²⁶X²⁷S
X²⁸X²⁹X³⁰PASATX³¹NADVRYARNEDFDAAMKTLEERAQQKKLPEAD
X³²KX³³IVTRGRPAFNAGEGGKX³⁴X³⁵X³⁶DKAX³⁷AX³⁸X³⁹KEAGGT
LGVEERTGGGTDAAYAALSGKPVIESLGLPGFGYHSDKAEYVDISAIPRR
LYMAARLIMDLGAGK

wherein X⁰ is optionally a targeting moiety such as an antibody domain;
X¹ is I, T; X² is L, A; X³ is M, K; X⁴ is L, I; X⁵ is Y, T; X⁶ is I, A; X⁷ is V, A; X⁸ is L, A;
X⁹ is L, T, A; X¹⁰ is K, T; X¹¹ is L, M, A; X¹² is L, T, A; X¹³ is L, G, T; X¹⁴ is I, T, A;
X¹⁵ is V, A; X¹⁶ is L, T, G; X¹⁷ is F, A; X¹⁸ is L, A; X¹⁹ is L, A; X²⁰ is V, A; X²¹ is L, A;
X²² is V, T, A; X²³ is L, A; X²⁴ is W, A, H; X²⁵ is I, A; X²⁶ is N, T, A; X²⁷ is V, T;
X²⁸ is N, T, A; X²⁹ is I, T, A; X³⁰ is I, T, A; X³¹ is L, T, A, I; X³² is V, A; X³³ is V, A;
X³⁴ is K, T; X³⁵ is L, A; X³⁶ is V, A; X³⁷ is V, S, T, A; X³⁸ is Y, T, A; X³⁹ is Y, S, T, A;
   and whereby simultaneously X¹=I, X²=L, X³=M, X⁴=L, X⁵=Y, X⁶=I, X⁷=V, X⁸=L, X⁹=L, X¹⁰=K, X¹¹=L, X¹²=L, X¹³=L, X¹⁴⁼I, X¹⁵=V, X¹⁶=L, X¹⁷=F, X¹⁸=L, X¹⁹=L, X²⁰=V, X²¹=L, X²²=V, X²³=L, X²⁴=W, X²⁵=I, X²⁶=N, X²⁷=V, X²⁸=N, X²⁹=I, X³⁰=I, X³¹=L, X³²=V, X³³=V, X³⁴=K, X³⁵=L, X³⁶=V, X³⁷=V, X³⁸=Y and X³⁹=Y are excluded.

Modified CPG2 proteins according to the above structure are an embodiment of the present invention. A modified CPG2 protein has been produced and has demonstrated reduced ability to elicit a proliferative response in human T-cells cultured in vitro (detailed in Example 6). Such data is concordant with a modified CPG2 protein having a reduced immunogenic potential in vivo.

It is recognized that the native CPG2 enzyme forms a homodimer and requires zinc ions for activity. It is an object of the present invention to produce a modified CPG2 molecule which contains a reduced number of T-cell epitopes or sequences able to bind to MHC class II or able to bind to a T-cell in association with an MHC class II molecule and which also preferably is able to form a homodimer and bind zinc ions.

Whilst it is recognized that the native CPG2 enzyme forms a homodimer and requires zinc ions for activity, it is most desired to provide a modified CPG2 molecule in which there is a reduced or absent potential to induce an immune response upon administration to a human subject and said modified CPG2 molecule with such a desired property may be compromised in its ability to form a homodimer and or bind zinc ions but retain a degree of enzyme activity with respect to its ability to convert a prodrug to an active drug. Such a molecule falls under the scope of the present invention.

A modified CPG2 molecule of the present invention may not form a homodimer and sequence modification resulting in a monomeric CPG2 but with a desired enzymatic activity and having none or at least a reduced number of T-cell epitopes or sequences able to bind to MHC class II or able to bind to a T-cell in association with an MHC class II molecule is equally also a desired object of the present invention.

It is recognized that a modified CPG2 molecule that is not able to form a homodimer of itself and may not have the desired enzymatic activity as monomer in solution could none the less have activity restored in part or in totality should two or more molecules of the modified CPG2 be brought into proximity. Such a molecule should it also have a reduced number of T cell epitopes or sequences able to bind to MHC class II or able to bind to a T cell in association with an MHC class II molecule equally falls under the scope of the present.

Such a situation where two of more modified CPG2 molecules are brought into proximity to thereby re-constitute an enzymatic activity could be achieved by genetic engineering means for example by fusion of the CPG2 molecules to domains from a second protein able to facilitate or engage in dimeric or other degrees of binding interaction. Examples of such domains include antibody constant regions such as the Fc domain of IgG or another immunoglobulin isotypes. Further examples include antibody V-region domains or the b-zip motif exemplified in proteins such as FOS and JUN. Other recognized protein domains may equally be contemplated. It may also be expected that a protein linker domain connecting two CPG2 moieties as a single recombinant fusion protein may also achieve the effect of bringing together into suitable proximity said CPG2 moieties.

The formation of modified CPG2 complexes with non-protein compounds including synthetic water soluble polymers such as hydroxypropylmethacrylamide or polystyrene-co-maleic acid or others may also be contemplated. Equally liposomal or carbohydrate preparations may be considered and conjugated with a modified CPG2 for the purpose of restoring or providing a degree of enzyme activity to the complex which otherwise would not be present were said CPG2 moieties not in close or forced proximity.

It is an objective of the present invention to produce a modified CPG2 molecule with a reduced or absent potential to induce an immune response upon administration to a human host. Within this objective it is also desired to retain the functional activity of the molecule with respect to its ability to catalyse the conversion of a prodrug to an active drug. It is preferred that the active drug is at least one order of magnitude more toxic to the desired target cell than the prodrug and it is most preferred that the active drug will be greater than one order of magnitude more toxic. Suitable prodrugs include nitrogen mustard prodrugs and other compounds as those described in WO88/07378; WO89/10140; WO90/02729; WO91/03460; EP-A-540263; WO94/02450; WO95/02420; WO95/03830 or U.S. Pat. No., 6,004,550 which are incorporated herein by reference. Any other compound able to undergo conversion by the modified CPG2 of the present invention and able to achieve a suitable toxicity profile may be contemplated for use in combination with the modified CPG2 of the present invention.

In as far as this invention relates to modified CPG2, compositions containing such modified CPG2 proteins or fragments of modified CPG2 proteins and related compositions should be considered within the scope of the invention. In another aspect, the present invention relates to nucleic acids encoding modified CPG2 entities. In a further aspect the present invention relates to methods for therapeutic treatment of humans using the modified CPG2 proteins. In this aspect the modified CPG2 protein may be linked with an antibody molecule or fragment of an antibody molecule. The linkage may be by means of a chemical cross-linker or the CPG2-antibody may be produced as a recombinant fusion protein. The fusion molecule may contain the modified CPG2 domain with antibody domain orientated towards the N-terminus of the fusion molecule although the opposite orientation may be contemplated.

Desired antibody specificities for linkage to the modified CPG2 molecule of the present include those directed towards the carcinoembryonic antigen as exemplified by numerous antibodies including MFE23 [Chester, K. A. et al (1994) Lancet 343: 455], A5B7 [WO92/010159], T84.66 [U.S. Pat. No., 5,081,235] MN-14 [Hansen, H. J. et al (1993) Cancer 71: 3478-3485], COL-1 [U.S. Pat. No., 5,472,693] and others. Other desired specificities include antibodies directed to non-internalizing antigens and this may include antigens such as the 40 kDa glycoprotein antigen as recognized by antibody KS1/4 [Spearman et al (1987) J. Pharmacol. Exp. Therapeutics 241: 695-703] and other antibodies. Other antigens such as the epidermal growth factor receptor (HER1) or related receptors such as HER2 may be selected including anti-GD2 antibodies such as antibody 14.18 [U.S. Pat. No., 4,675,287; EP 0 192 657], or antibodies to the prostate specific membrane antigen [U.S. Pat. No., 6,107,090], the IL-2 receptor [U.S. Pat. No., 6,013,256], the A33 antigen [Heath, J. K. et al (1997) Proc. Natl, Acad. Sci U.S.A. 94: 469-474], the Lewis Y determinant, mucin glycoproteins or others may be contemplated.

In all instances where a modified CPG2 protein is made in fusion with an antibody sequence it is most desired to use antibody sequences in which T cell epitopes or sequences able to bind MHC class II molecules or stimulate T cells or bind to T cells in association with MHC class II molecules have been removed.

A further embodiment of the present invention, the modified CPG2 protein may be linked to a non-antibody protein yet a protein able to direct a specific binding interaction to a particular target cell. Such protein moieties include a variety of polypeptide ligands for which there are specific cell surface receptors and include therefore numerous cytokines, peptide and polypeptide hormones and other biological response modifiers. Prominent examples include such proteins as vascular epithelial growth factor, epidermal growth factor, heregulin, the interleukins, interferons, tumor necrosis factor and other protein and glycoprotein molecules. Fusion proteins of these and other molecules with CPG2 of the present invention may be contemplated and may comprise the modified CPG2 moiety in either the N-terminal or C-terminal orientation with respect to the protein ligand domain. Equally, chemical cross-linking of the purified ligand to the modified CPG2 protein may be contemplated and within the scope of the present invention.

In a further embodiment the modified CPG2 protein of the present invention may be used as a complex containing a water soluble polymer such as hydroxypropylmethacrylamide or other polymers where the modified CPG2 protein is in covalent attachment to the polymer or in a non-covalent binding interaction with the polymer. Such an embodiment may additionally include an antigen binding domain such as and antibody or a fragment of an antibody in combination with the polymer CPG2 complex.

In a further embodiment of the present invention the gene for the modified CPG2 enzyme may itself be used as a therapeutic entity such as the gene directed enzyme prodrug strategy and may include linkage to a tissue specific promoter sequence within the vector or may be expressed from a promoter of viral origin within the vector or the vector itself may be of viral origin or able to be packaged within viral particles able to infect cells. The invention will now be illustrated but not limited, by the following examples. The examples refer to the following drawings:

FIG. 1 provides a listing of the synthetic peptides used in the naïve T-cell proliferation assay according to the method of EXAMPLE 2. ID#=the identification number of each peptide tested; Donor #=identifies donor PBMC cultures in which a stimulation index of >0.95 was scored for the particular peptide; Sequence=the peptide sequence in single letter code.

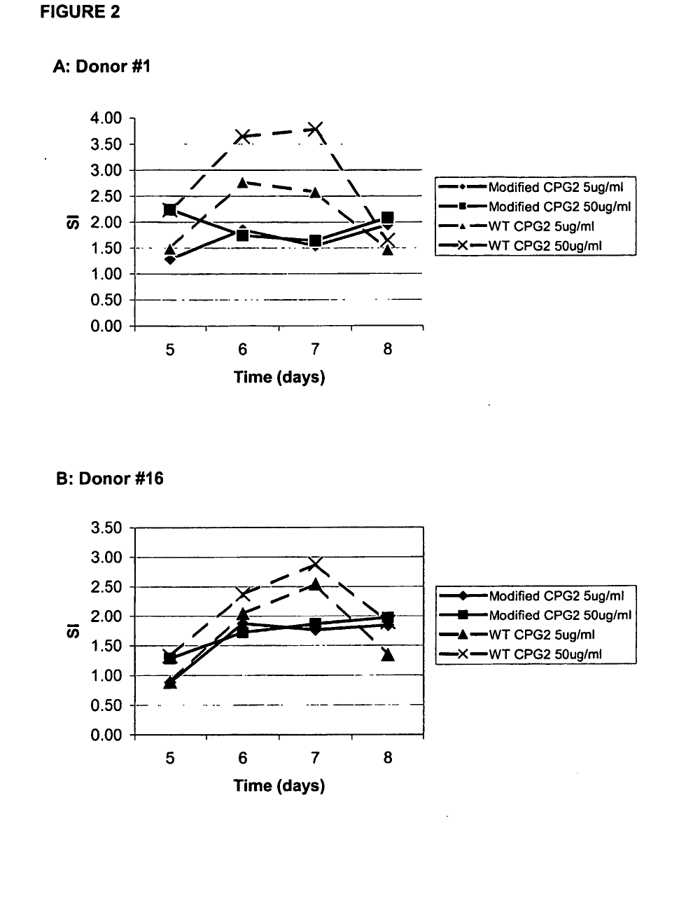

FIG. 2 provides a plots depicting the results of an immunogenicity assay using naïve human PBMC cultured in the presence of differing concentrations of wild-type or modified CPG2 protein. The graphs show the results from two responsive donor PBMC samples. Panel A=donor #1. Panel B=donor #16. SI=stimulation index.

### EXAMPLE 1

Identification of Potential MHC Class II Ligands in the CPG2 Protein Sequence by Computational Means.

The scheme for the analysis of peptide sequences with potential to act as MHC class II binding ligands has been described in detail previously [WO 02/069232]. A software tool using this procedure has been developed and applied to the analysis of the CPG2 protein sequence. In brief, the software was built from a number of elements including a library of model MHC class II molecules, produced to cover a wide number of allotypic variants extant in the human population and a library of peptide backbone structures, produced to encompass theoretical and known backbone conformations. Using these elements, a large data set was generated based on the results of docking each backbone conformation within each model MHC allotype binding groove. The data set encompasses also the best side-chain conformation for all possible amino acids at the given position. The interatomic distances between the peptide side-chains (in the optimum conformation) and the MHC protein are stored in this dataset. A test peptide from the protein of interest is analyzed by adding its sequence of side-chains to all backbones and then retrieving the data sets for the optimum side-chain conformations and thus calculating a “peptide score” for each backbone. The best score is selected for display and the process repeated for each of the available MHC model structures.

The algorithm has been applied to the analysis of the entire CPG2 protein sequence. The analysis identifies multiple 13mer peptide sequences which are potential T-cell epitopes by their being predicted MHC class II ligands for one or more allotypes. These peptides are shown in table 1 wherein the peptide sequences are depicted using single letter code.

### EXAMPLE 2

Identification of T-Cell Epitopes Using Synthetic Peptides and Naïve Human PBMC In Vitro Proliferation Assays.

The interaction between MHC, peptide and T-cell receptor (TCR) provides the structural basis for the antigen specificity of T-cell recognition. T-cell proliferation assays test the binding of peptides to MHC and the recognition of MHC/peptide complexes by the TCR. In vitro T-cell proliferation assays of the present example, involve the stimulation of peripheral blood mononuclear cells (PBMCs), containing antigen presenting cells (APCs) and T-cells. Stimulation is conducted in vitro using synthetic peptide antigens, and in some experiments whole protein antigen. Stimulated T-cell proliferation is measured using ³H-thymidine (³H-Thy) and the presence of incorporated ³H-Thy assessed using scintillation counting of washed fixed cells.

Donated cells were obtained from the National Blood Service (Addenbrooks Hospital, Cambridge, UK). Ficoll-paque was obtained from Amersham Pharmacia Biotech (Amersham, UK). Serum free AIM V media for the culture of primary human lymphocytes and containing L-glutamine, 50□g/ml streptomycin, 10□g/ml gentomycin and 0.1% human serum albumin was from Gibco-BRL (Paisley, UK). Synthetic peptides were obtained from Eurosequence (Groningen, The Netherlands) and Babraham Technix (Cambridge, UK).

Erythrocytes and leukocytes were separated from plasma and platelets by gentle centrifugation of buffy coats. The top phase (containing plasma and platelets) was removed and discarded. Erythrocytes and leukocytes were diluted 1:1 in phosphate buffered saline (PBS) before layering onto 15 ml ficoll-paque (Amersham Pharmacia, Amersham UK). Centrifugation was done according to the manufacturers recommended conditions and PBMCs were harvested from the serum+PBS/ficoll paque interface. PBMCs were mixed with PBS (1:1) and collected by centrifugation. The supernatant was removed and discarded and the PBMC pellet re-suspended in 50 ml PBS. Cells were again pelleted by centrifugation and the PBS supernatant discarded. Cells were re-suspended using 50 ml AIM V media and at this point counted and viability assessed using trypan blue dye exclusion. Cells were again collected by centrifugation and the supernatant discarded. Cells were re-suspended for cryogenic storage at a density of 3×10⁷ per ml. The storage medium was 90%(v/v) heat inactivated AB human serum (Sigma, Poole, UK) and 10% (v/v) DMSO (Sigma, Poole, UK). Cells were transferred to a regulated freezing container (Sigma) and placed at −70° C. overnight. When required for use, cells were thawed rapidly in a water bath at 37° C. before transferring to 10 ml pre-warmed AIM V medium.

The tissue types for all PBMC samples were assayed using a commercially available reagent system (Dynal, Wirral, UK). Assays were conducted in accordance with the suppliers recommended protocols and standard ancillary reagents and agarose electrophoresis systems. The tissue types of the panel of 20 donor PBMC samples selected for the CPG2 epitope analysis are identified in table 6 (below) and were chosen to provide a wide spectrum of allotypes.

TABLE 6
Tissue types of donor panel

| No | DR typing |
|---|---|
| 1 | DRB1*01, DRB1*08 |
| 2 | DRB1*13, DRB1*0103 and DRB3 |
| 3 | DRB1*03, DRB1*14, DRB3 |
| 4 | DRB1*01, DRB1*03, DRB3 |
| 5 | DRB1*01, DRB1*04, DRB4*01 |
| 6 | DRB1*01, DRB1*07, DRB4*01 |
| 7 | DRB1*03, DRB1*04, DRB3, |
| | DRB4*01 |
| 8 | DRB1*07, DRB1*15 and DRB4*01, |
| | DRB5 |
| 9 | DRB1*01, DRB1*11 and DRB3 |
| 10 | DRB1*04, DRB1*08, DRB4 |
| 11 | DRB1*03, DRB1*15, DRB3, DRB5 |
| 12 | DRB1*04 AND DRB1*08, 11 OR 13, |
| | DRB3, DRB4*01 |
| 13 | DRB1*04, DRB1*16, DRB4*01, |
| | DRB5 |
| 14 | DRB1*12, DRB1*15, DRB3, DRB5 |
| 15 | DRB1*01, DRB1*04, DRB4*01 |
| 16 | DRB1*03, DRB1*09, DRB3, |
| | DRB4*01 |
| 17 | DRB1*13, DRB1*15, DRB3, DRB5 |
| 18 | DRB1*10, DRB1*13, DRB3 |
| 19 | DRB1*11, DRB1*15, DRB3, DRB5 |
| 20 | DRB1*13, DRB3 |

PBMC were stimulated with protein and peptide antigens in a 96 well flat bottom plate at a density of 2×10⁵ PBMC per well. PBMC were incubated for 7 days at 37° C. before pulsing with ³H-Thy (Amersham-Pharmacia, Amersham, UK). For the present study, synthetic peptides (15mers) spanning the entire CPG2 sequence and including an additional C-terminal 6-His tag were produced. Each peptide overlapped each successive peptide in the sequence by 12 residues, i.e., each peptide incremented from the next in the sequence by 3 residues. The peptide sequences and identification numbers are shown in FIG. 1. Each peptide was screened individually against PBMC's isolated from 20 naïve donors. Two control peptides C32 (PKYVKQNTLKLAT) and C49 (KVVDQIKKISKPVQH) that have previously been shown to be immunogenic and a potent non-recall antigen KLH were used in each donor assay. Peptides were dissolved in DMSO to a final concentration of 10 mM, these stock solutions were then diluted 1/500 in AIM V media (final concentration 20 μM). Peptides were added to a flat bottom 96 well plate to give a final concentration of 2 and 10 μM in a 100 μl. The viability of thawed PBMC's was assessed by trypan blue dye exclusion, cells were then re-suspended at a density of 2×10⁶ cells/ml, and 100 μl (2×10⁵ PBMC/well) was transferred to each well containing peptides. Triplicate well cultures were assayed at each peptide concentration. Plates were incubated for 7 days in a humidified atmosphere of 5% CO₂ at 37° C. Cells were pulsed for 18-21 hours with 1 μCi ³H-Thymidine per well before harvesting onto filter mats. CPM values were determined using a Wallac microplate beta top plate counter (Perkin Elmer). Results were expressed as stimulation indices (SI), where SI=CPM Test Peptide/CPM untreated control.

Mapping T cell epitopes in the CPG2 sequences using the naïve T cell proliferation assay resulted in the identification of several immunogenic regions. Peptides with significant stimulation indices in individual donors are listed in FIG. 1. The results of the naïve T-cell proliferation assay can be used to compile an epitope map of the CPG2 protein. In general, in compilation of such a map, an SI>1.95 is taken as a positive response.

### EXAMPLE 3

Production of CPG2 Gene.

The original sequence of CPG2 was taken from that of Pseudomonas sp. Strain RS-16 (gene bank accession no. AE002078). The protein sequence of 390 amino acids was back-translated to give a DNA sequences of 1170 nucleotides. Back-translation was done using commercially available software (DNAstar, Madison, Wis., USA) and the sequence compiled based on the most frequently used codons for E. coli. The sequence was used to design a set of 24 synthetic oligonucleotides. The oligonucleotides ranged in size from 50 to 83 nucleotides in length and were designed to have overlapping temini of 19 to 25 nucleotides. The gene was designed also to have an Asc I site at the 5′ end and a Sac I site at the 3′ end to allow cloning into a plasmid vector. The oligonucleotides are listed in table 7.

TABLE 7
Synthetic oligonucleotide sequences

| | |
|---|---|
| Name | Sequence |

| | |
|---|---|
| OL549 | CAGAAACGTGACAACGTTCTGTTCCAGGCTGCTACCGACGAACA |
| | GCCGGCTGTTATCAAAACCCTGGAAAAAC |
| OL550 | GAAGTTACCAGCAGCAGCGATACCTTCAGCGTCACCGGTACCGG |
| | TTTCGATGTTAACCAGTTTTTCCAGGGTTTTGATAAC |
| OL551 | GTATCGCTGCTGCTGGTAACTTCCTGGAAGCTGAACTGAAAAAC |
| | CTGGGTTTCACCGTTACCCGTTCTAAATCTGCTGGTC |
| OL552 | CAGCAGGTTTTTACCACCACGACCTTTGATTTTACCAACGATGT |
| | TGTCACCAACAACCAGACCAGCAGATTTAGAACGGG |
| OL553 | CGTGGTGGTAAAAACCTGCTGCTGATGTCTCACATGGACACCGT |
| | TTACCTGAAAGGTATCCTGGCTAAAGCTC |
| OL554 | GTCGTCAGCGATACCCGGACCGTAAGCTTTGTCACCTTCAACAC |
| | GGAACGGAGCTTTAGCCAGGATACCTTTC |
| OL555 | GTCCGGGTATCGCTGACGACAAAGGTGGTAACGCTGTTATCCTG |
| | CACACCCTGAAACTGCTGAAAGAATACGGTGTTC |
| OL556 | GAACCGAAAGAACCTTTTTCTTCGTCGGTGTTGAACAGAACGGT |
| | GATGGTACCGTAGTCACGAACACCGTATTCTTTCAGCAG |
| OL557 | GAAGAAAAAGGTTCTTTCGGTTCTCGTGACCTGATCCAGGAAGA |
| | AGCTAAACTGGCTGACTACGTTCTGTCTTTCG |
| OL558 | CTGAACGTAAGCGATACCAGAGGTACCCAGAGACAGTTTTTCGT |
| | CACCAGCAGAGGTCGGTTCGAAAGACAGAACGTAGTCAG |
| OL559 | CTCTGGTATCGCTTACGTTCAGGTTAACATCACCGGTAAAGCTT |
| | CTCACGCTGGTGCTGCTCCGGAACTGGGTGTTAACGCTC |
| OL560 | GTTTTTAGCTTTGTCGTCGATGTTCATGGTACGCAGAACCAGGT |
| | CAGAAGCTTCAACCAGAGCGTTAACACCCAGTTCCG |
| OL561 | CATCGACGACAAAGCTAAAAACCTGCGTTTCAACTGGACCATCG |
| | CTAAAGCTGGTAACGTTTCTAACATCATCCCG |
| OL562 | CATAGCAGCGTCGAAGTCTTCGTTACGAGCGTAACGAACGTCAG |
| | CGTTCAGGGTAGCAGAAGCCGGGATGATGTTAGAAACG |
| OL563 | GAAGACTTCGACGCTGCTATGAAAACCCTGGAAGAACGTGCTCA |
| | GCAGAAAAAACTGCCGGAAGCTGACGTTAAAG |
| OL564 | CTTCACCAGCGTTGAAAGCCGGACGACCACGGGTAACGATAACT |
| | TTAACGTCAGCTTCCGGCAG |
| OL565 | CGGCTTTCAACGCTGGTGAAGGTGGTAAAAAACTGGTTGACAAA |
| | GCTGTTGCTTACTACAAAGAAGCTGGTGGTAC |
| OL566 | GACAGAGCAGCGTAAGCAGCGTCGGTACCACCACCGGTACGTTC |
| | TTCAACACCCAGGGTACCACCAGCTTCTTTGTAGTAAG |
| OL567 | GCTGCTTACGCTGCTCTGTCTGGTAAACCGGTTATCGAATCTCT |
| | GGGTCTGC |
| OL568 | CGTATTCAGCTTTGTCAGAGTGGTAACCGAAACCCGGCAGACCC |
| | AGAGATTCGATAAC |
| OL569 | CACTCTGACAAAGCTGAATACGTTGACATCTCTGCTATCCCGCG |
| | TCGTCTGTACATGGCTGCTC |
| OL570 | TTTACCAGCACCCAGGTCCATGATCAGACGAGCAGCCATGTACAG |
| | ACGAC |
| OL571 | AAAAAAGAGCTCTTTACCAGCACCCAGGTCCATGATC |
| OL572 | AAAAGGCGCGCCGCAGAAACGTGACAACGTTCTGTTCCAG |

The gene was assembled by polymerase chain reaction (PCR). A set of four different PCR mixes (A, B, C, D) were compiled featuring different sets of olignucleotides as identified below. In all instances the primers driving the reaction were present in higher concentration within each mix (50 pmol), and are shown below underlined:
Mix A) OL549+OL550+OL551+OL552
Mix B) OL553+OL554+OL555+OL556+OL557+OL558
Mix C) OL559+OL560+OL561+OL562+OL563+OL564
Mix D) OL565+OL566+OL567+OL568+OL569+OL570

The complete CPG2 gene was formed in a joining reaction using the purified products of the above reactions and driven by flanking primers OL571 and OL572 to introduce the cloning sites. The PCRs were conducted using hi fidelity polymerase (Promega, Southampton, UK) and a reaction buffer supplied with the enzyme. The reactions were cycled using a thermal cycler running the program detailed below:

PCR Cycling Conditions:

| Stage | Step | Temperature (° C.) | Time (sec) | No of Cycles |
|---|---|---|---|---|
| 1 | 1 | 94 | 120 | 1 |
| 2 | 1 | 94 | 15 | 10 |
| 2 | 2 | 45-60 (gradient) | 60 | 10 |
| 2 | 3 | 72 | 45 | 10 |
| 3 | 1 | 94 | 15 | 25 |
| 3 | 2 | 60 | 30 | 25 |
| 3 | 3 | 72 | 45 | 25 |
| 4 | 1 | 72 | 420 | 1 |
| 4 | 2 | 4 | hold | 1 |

The assembled CPG2 gene was subcloned into pGEM and the sequence confirmed by sequence analysis. To test the activity of CPG2, cells containing the CPG2 gene were plated out on to LB agar containing 0.1% folic acid (folic acid was dissolved in 1M NaOH to make a 10% stock). These were then incubated at 37° C., colonies that had CPG2 activity were identified by a yellow halo. The halo is the pteroic acid that is precipitated when the folic acid is hydrolyzed.

### EXAMPLE 4

Site-Directed Mutagenesis of CPG2 Gene

The cloned active CPG2 gene was used as a template for the development of mutated variants of the gene using the QuickChange™ Site-Directed Mutagenesis Kit (Stratagene, LaJolla, Calif.). A high fidelity thermostable polymerase is used to extend pairs of oligonucleotide primers, which are complementary to opposite strands of the vector and contain the desired mutation. Incorporation of the primers results in a mutated vector containing staggered nicks. Parental DNA is digested using DpnI endonuclease which is specific for methylated and hemimethylated DNA and the nicked vector DNA incorporating the desired mutations is then transformed into competent E. coli cells. Sixteen pairs of oligonucleotide primers designed to introduce a point mutation each in the template, were used. Oligonucleotide sequences are shown in Table 8.

TABLE 8

Oligonucleotide primers used to introduce
mutations to the CPG2 template gene. The
sequence, length and the mutation introduced
is shown.

| | | | |
|---|---|---|---|
| Name | Substitutions | Primer Sequence | Length |

| | | | | |
|---|---|---|---|---|
| 1 | OL573 | L93I | CCGTTTACATAAAAGGTA | 18 |
| | | (A4) | | |
| 2 | OL574 | L128T | ACACCACGAAACTGCTG | 17 |
| | | (B5) | | |
| 3 | OL575 | K129T | CCCTGACACTGCTGAAAG | 18 |
| | | (B6) | | |
| 4 | OL576 | L130M | CTGAAAATGCTGAAAGAA | 18 |
| | | (B7) | | |
| 5 | OL577 | L131T | GAAACTGACGAAAGAATA | 18 |
| | | (B8) | | |
| 6 | OL578 | I161T | ACCTGACCCAGGAAGAA | 17 |
| | | (C2) | | |
| 7 | OL579 | V217T | CGCTCTGACTGAAGCCTC | 18 |
| | | (E4) | | |
| 8 | OL580 | N247T | AAGCTGGAACCGTTTCTA | 18 |
| | | (F4) | | |
| 9 | OL581 | N250T | AACGTTTCTACCATCATC | 18 |
| | | (F6) | | |
| 10 | OL582 | I251T | GTTTCTAACACCATCCC | 17 |
| | | (F7) | | |
| 11 | OL583 | I252T | TAACATCACCCCGGCTTC | 18 |
| | | (F8) | | |
| 12 | OL584 | L258T | TCTGCTACCACGAACGCT | 18 |
| | | (F9) | | |
| 13 | OL585 | K310T | GTGGTAAAACACTGGTTG | 18 |
| | | (G3) | | |
| 14 | OL586 | V316S | ACAAAGCATCTGCTTACT | 18 |
| | | (H3) | | |
| 15 | OL587 | Y318T | TGTTGCTACCTACAAAGA | 18 |
| | | (H4) | | |
| 16 | OL588 | Y319S | TTGCTTACTCCAAAGAA | 17 |
| | | (H5) | | |
| 1 | OL589 | L93I | TACCTTTTATGTAAACGG | 18 |
| | | (A4) R | | |
| 2 | OL590 | L128T | CAGCAGTTTCGTGGTGT | 17 |
| | | (B5) R | | |
| 3 | OL591 | K129T | CTTTCAGCAGTGTCAGGG | 18 |
| | | (B6) R | | |
| 4 | OL592 | L130M | TTCTTTCAGCATTTTCAG | 18 |
| | | (B7) R | | |
| 5 | OL593 | L131T | TATTCTTTCGTCAGTTTC | 18 |
| | | (B8) R | | |
| 6 | OL594 | I161T | TTCTTCCTGGGTCAGGT | 17 |
| | | (C2) R | | |
| 7 | OL595 | V217T | GAGGCTTCAGTCAGAGCG | 18 |
| | | (E4) R | | |
| 8 | OL596 | N247T | TAGAAACGGTTCCAGCTT | 18 |
| | | (F4) R | | |
| 9 | OL597 | N250T | GATGATGGTAGAAACGTT | 18 |
| | | (F6) R | | |
| 10 | OL598 | I251T | GGGATGGTGTTAGAAAC | 17 |
| | | (F7) R | | |
| 11 | OL599 | I252T | GAAGCCGGGGTGATGTTA | 18 |
| | | (F8) R | | |
| 12 | OL600 | L258T | AGCGTTCGTGGTAGCAGA | 18 |
| | | (F9) R | | |
| 13 | OL601 | K310T | CAACCAGTGTTTTACCAC | 18 |
| | | (G3) R | | |
| 14 | OL602 | V316S | AGTAAGCAGATGCTTTGT | 18 |
| | | (H3) R | | |
| 15 | OL603 | Y318T | TCTTTGTAGGTAGCAACA | 18 |
| | | (H4) R | | |
| 16 | OL604 | Y319S | TTCTTTGGAGTAAGCAA | 17 |
| | | (H5) R | | |

### EXAMPLE 5

Expression and Purification of Recombinant CPG2

Mutant CPG2 genes were cloned into an expression vector utilizing the AscI/SacI restriction sites flanking the coding region. Ligation mixtures were used to transform E. Coli cells. Several ampicillin resistant clones were selected on LB plates containing 50-100 μg/ml ampicillin. These were analyzed for the presence and orientation of the recombinant insert and to ensure that it is in frame with the His-tag. After verification of the correct orientation, cells were grown at 37° C., induced for expression and harvested by centrifugation. Expression was confirmed by analysis of lyzed cell samples in SDS-PAGE gels and stained with Coomassie Blue. Expression was scaled up to 50 ml cell cultures for subsequent purification of recombinant protein.

The 6×His tagged CPG2 protein was purified using ProBond™ Purification System (Invitrogen, Carlsbad, Calif.) and protocols recommended by the supplier. Briefly, cells were harvested from 50 ml cultures by centrifugation, re-suspended in Binding Buffer and lyzed. Lysates were allowed to bind to Purification Column resin under gentle agitation for 60 minutes. Resin was washed and recombinant protein was eluted using Elusion Buffer. Samples were analyzed using SDS-PAGE and Coomassie staining as previously. The concentration of the purified protein was determined using spectrophotometry.

### EXAMPLE 6

Demonstration of Reduced Immunogenic Potential in Modified CPG2 Protein Using Human PBMC In Vitro Proliferation Assay.

A modified protein was prepared according to the method of examples 3-5. The modified protein contained multiple substitutions from the wild-type sequence. Positive control protein was wild-type CPG2.

For T cell proliferation assays 4×10⁶ PBMC (per well) from healthy donors were incubated with unmodified and modified antibodies in 2 ml bulk cultures (in 24 well plates). Each donor culture was treated with modified and unmodified CPG2 at 5 and 50 μg/ml. In addition an untreated control bulk culture was maintained enabling stimulation indexes to be determined. At days 5, 6, 7 and 8 cells for each bulk culture were gently agitated and 50 μl samples removed in triplicate for determination of the proliferation index. The 50 μl sample aliquots were each transferred to 3 wells of a U-bottom 96 well plate. Fresh AIM V media (130 μl) was added to each of the 96 wells. Cells were pulsed (for 18-21 hours) with 1 μCi [³H]Thymidine per well diluted in a total volume of 20 ul AIM V media. The total volume for each culture was 200 μl. CPM values were collected using a beta-plate reader and the stimulation index for each time point determined as per Example 2.

The SI was plotted for each time point and treatment. In responsive donors, treatment with wild-type CPG2 results in a significant proliferative response with a peak at day 7. In the same donors, treatment with the modified CPG2 composition of the present invention does not result in a significant proliferative response. This results indicate a reduced immunogenic potential in the modified CPG2 protein. Plots from responsive donors #1 and #16 are provided in FIG. 2.

1. A modified bacterial enzyme carboxypeptidease G2 (CPG2) being substantially non-immunogenic or less immunogenic than any non-modified CPG2 having essentially the same biological specificity when used in vivo, and comprising specific amino acid residues having alterations compared with the non-modified parental enzyme, wherein said alterations cause a reduction or an elimination of one or more of T-cell epitope sequences, which act in the parental enzyme as MHC class II binding ligands and stimulate T-cells.

2. A modified CPG2 molecule according to claim 1, wherein said alterations are made at one or more positions within following strings of contiguous amino acid residues from the CPG2 wild-type sequence:
A = VGKIKGRGGKNLLLMSHMDTVYLKGILAK;
B = KAYGPGIADDKGGNAVILHTLKLLKEYG;
C = LFNTDEEKGSFGSRDLIQEEA;
D = KLADYVLSFEPTSAGDEKLSLGTSG;
E = VNITGKASHAGAAPELGVNALVEASDL;
F = KAKNLRFNWTIAKAGNVSNIIPASATLNAD;
G = ADVKVIVTRGRPAFNAGEGGKKLVDKA;
H = KKLVDKAVAYYKEAGG;
I = YKEAGGTLGVEERTGGG;
J = TDAAYAALSGKPVIESLGLPGFGY
K = LEKLVNIETGTGDAE;

3. A modified CPG2 molecule according to claim 1, wherein said T-cell epitope sequences are 13mer or 15mer peptides and are selected from any of Table 1 or Table 2.

4. A modified CPG2 molecule according to any of the claims 1 to 3, wherein said alterations are substitutions of 1-9 amino acid residues.

5. A modified CPG2 molecule of claim 3, wherein said substitutions are selected from any of Table 4 or Table 5.

6. A modified CPG2 molecule according to any of the claims 1 to 5, comprising one or more further alterations of amino acid residues, wherein said alterations are conducted to restore biological activity of the molecule.

7. A molecule having the biological activity of bacterial enzyme carboxypeptidease G2 (CPG2) and the following amino acid sequence:
X⁰QKRDNVLFQAATDEQPAVIKTLEKLVNIETGTGDAEGIAAAGNFLEAELKNLGFTVT
RSKSAGLVVGDNIVGKX¹KGRGGKNLX²LX³SHMDTVYX⁴KGILAKAPFRVEGDKAX⁵GP
GX⁶ADDKGGNAX⁷IX⁸HTX⁹X¹⁰X¹¹X¹²KEYGVRDYGTITVX¹³FNTDEEKGSFGSRDLX¹⁴Q
EEAKLADYX¹⁵X¹⁶SX¹⁷EPTSAGDEKX¹⁸SLGTSGIAYVQVNITGKASHAGAAPEX¹⁹GX²⁰
NAX²¹X²²EASDLVLRTMNIDDKAKNX²³RFNX²⁴TX²⁵AKAGX²⁶X²⁷SX²⁸X²⁹X³⁰PASATX³
¹NADVRYARNEDFDAAMKTLEERAQQKKLPEADX³²KX³³IVTRGRPAFNAGEGGKX³⁴X³⁵
X³⁶DKAX³⁷AX³⁸X³⁹KEAGGTLGVEERTGGGTDAAYAALSGKPVIESLGLPGFGYHSDKA
EYVDISAIPRRLYMAARLIMDLGAGK,
wherein X⁰ is optionally a targeting moiety such as an antibody domain and
X¹ is I, T; X² is L, A; X³ is M, K; X⁴ is L, I; X⁵ is Y, T; X⁶ is I, A; X⁷ is V, A;
X⁸ is L, A; X⁹ is L, T, A; X¹⁰ is K, T; X¹¹ is L, M, A; X¹² is L, T, A; X¹³ is L, G, T;
X¹⁴ is I, T, A; X¹⁵ is V, A; X¹⁶ is L, T, G; X¹⁷ is F, A; X¹⁸ is L, A; X¹⁹ is L, A;
X²⁰ is V, A; X²¹ is L, A; X²² is V, T, A; X²³ is L, A; X²⁴ is W, A, H; X²⁵ is I, A;
X²⁶ is N, T, A; X²⁷ is V, T; X²⁸ is N, T, A; X²⁹ is I, T, A; X³⁰ is I, T, A; X³¹ is L, T, A, I;
X³² is V, A; X³³ is V, A; X³⁴ is K, T; X³⁵ is L, A; X³⁶ is V, A; X³⁷ is V, S, T, A;
X³⁸ is Y, T, A; X³⁹ is Y, S, T, A;
and whereby simultaneously X¹=I, X²=L, X³=M, X⁴=L, X⁵=y, X⁶=I, X⁷=V, X⁸=L, X⁹=L, X¹⁰=K, X¹¹=L, X¹²=L, X¹³=L, X14=I, X¹⁵=V, X¹⁶=L, X¹⁷=F, X¹⁸=L, X¹⁹=L, X²⁰=V, X²¹=L, X²²=V, X²³=L, X²⁴=W, X²⁵=I, X²⁶=N, X²⁷=V, X²⁸=N, X²⁹=I, X³⁰=I, X³¹=L, X³²=V, X³³=V, X³⁴=K, X³⁵=L, X³⁶=V, X³⁷=V, X³⁸=Y and X³⁹=Y are excluded.

8. A modified CPG2 enzyme according to any of the claims 1 to 7, wherein when tested as a whole protein in a biological assay of induced cellular proliferation of human T-cells exhibits a stimulation index smaller than the parental enzyme tested in parallel using cells from the same donor wherein said index is taken as the value of cellular proliferation scored following stimulation by the protein and divided by the value of cellular proliferation scored in control cells not in receipt of protein and wherein cellular proliferation is measured by any suitable means.

9. A DNA sequence coding for a CPG2 molecule as defined in any of the claims 1 to 8.

10. A pharmaceutical composition comprising a modified CPG2 molecule of any of the preceding claims, optionally together with a pharmaceutically acceptable carrier, diluent or excipient.

11. A peptide molecule consisting of 9-15 consecutive amino acid residues, having a potential MHC class II binding activity and created from the primary sequence of non-modified CPG2 enzyme, whereby said peptide molecule has a stimulation index of at least 1.8 to 2 in a biological assay of cellular proliferation wherein said index is taken as the value of cellular proliferation scored following stimulation by a peptide and divided by the value of cellular proliferation scored in control cells not in receipt peptide and wherein cellular proliferation is measured by any suitable means.

12. A peptide molecule according to claim 11, wherein said stimulation index is greater than 2.

13. A peptide molecule of claim 11 or 12 selected from the group of contiguous T-cell epitope sequences as depicted in Table 1 or Table 2.

14. A modified peptide molecule deriving from the peptide molecule of any of the claims 11 to 13 by amino acid substitution, having a reduced or absent potential MHC class II binding activity expressed by a stimulation index of less than 2, whereby said index is taken as the value of cellular proliferation scored following stimulation by a peptide and divided by the value of cellular proliferation scored in control cells not in receipt peptide and wherein cellular proliferation is measured by any suitable means.

15. A modified peptide molecule of claim 14, wherein said stimulation index is less than 2.

16. Use of a peptide according to any of the claims 11 to 15 for the manufacture of a modified CPG2 enzyme having substantially no or less immunogenicity than any non-modified parental enzyme when used in vivo.

17. Use of a peptide according to any of the claims 11 to 13 for the purpose of vaccination of patients to reduce immunogenicity to CPG2 in vivo.

18. A DNA sequence coding for a peptide of any of the claims 11 to 15.

19. Pharmaceutical composition comprising a peptide of any of the claims 11 to 15, optionally together with a pharmaceutically acceptable carrier, diluent or excipient.


SEQUENCE LISTING (312 sequences)

SEQ ID NO: 1 - protein, 390 aa, homo sapiens
QKRDNVLFQAATDEQPAVIKTLEKLVNIETGTGDAEGIAAAGNFLEAELKNLGFTVTRSKSAGLVVGDNIVGKIKGRGGKNLLLMSHMDTVYLKGILAKAPFRVEGDKAYGPGIADDKGGNAVILHTLKLLKEYGVRDYGTITVLFNTDEEKGSFGSRDLIQEEAKLADYVLSFEPTSAGDEKLSLGTSGIAYVQVNITGKASHAGAAPELGVNALVEASDLVLRTMNIDDKAKNLRFNWTIAKAGNVSNIIPASATLNADVRYARNEDFDAAMKTLEERAQQKKLPEADVKVIVTRGRPAFNAGEGGKKLVDKAVAYYKEAGGTLGVEERTGGGTDAAYAALSGKPVIESLGLPGFGYHSDKAEYVDISAIPRRLYMAARLIMDLGAGK

SEQ ID NO: 2 - protein, 29 aa, homo sapiens
VGKIKGRGGKNLLLMSHMDTVYLKGILAK

SEQ ID NO: 3 - protein, 28 aa, homo sapiens
KAYGPGIADDKGGNAVILHTLKLLKEYG

SEQ ID NO: 4 - protein, 21 aa, homo sapiens
LFNTDEEKGSFGSRDLIQEEA

SEQ ID NO: 5 - protein, 25 aa, homo sapiens
KLADYVLSFEPTSAGDEKLSLGTSG

SEQ ID NO: 6 - protein, 27 aa, homo sapiens
VNITGKASHAGAAPELGVNALVEASDL

SEQ ID NO: 7 - protein, 30 aa, homo sapiens
KAKNLRFNWTIAKAGNVSNIIPASATLNAD

SEQ ID NO: 8 - protein, 27 aa, homo sapiens
ADVKVIVTRGRPAFNAGEGGKKLVDKA

SEQ ID NO: 9 - protein, 16 aa, homo sapiens
KKLVDKAVAYYKEAGG

SEQ ID NO: 10 - protein, 17 aa, homo sapiens
YKEAGGTLGVEERTGGG

SEQ ID NO: 11 - protein, 24 aa, homo sapiens
TDAAYAALSGKPVIESLGLPGFGY

SEQ ID NO: 12 - protein, 15 aa, homo sapiens
LEKLVNIETGTGDAE

SEQ ID NO: 13 - protein, 390 aa, Artificial Sequence
  Variant of human CPG2
QKRDNVLFQAATDEQPAVIKTLEKLVNIETGTGDAEGIAAAGNFLEAELKNLGFTVTRSKSAGLVVGDNIVGKXKGRGGKNLXLXSHMDTVYXKGILAKAPFRVEGDKAXGPGXADDKGGNAXIXHTXXXXKEYGVRDYGTITVXFNTDEEKGSFGSRDLXQEEAKLADYXXSXEPTSAGDEKXSLGTSGIAYVQVNITGKASHAGAAPEXGXNAXXEASDLVLRTMNIDDKAKNXRFNXTXAKAGXXSXXXPASATXNADVRYARNEDFDAAMKTLEERAQQKKLPEADXKXIVTRGRPAFNAGEGGKXXXDKAXAXXKEAGGTLGVEERTGGGTDAAYAALSGKPVIESLGLPGFGYHSDKAEYVDISAIPRRLYMAARLIMDLGAGK

SEQ ID NO: 14 - protein, 13 aa, homo sapiens
DNVLFQAATDEQP

SEQ ID NO: 15 - protein, 13 aa, homo sapiens
NVLFQAATDEQPA

SEQ ID NO: 16 - protein, 13 aa, homo sapiens
VLFQAATDEQPAV

SEQ ID NO: 17 - protein, 13 aa, homo sapiens
PAVIKTLEKLVNI

SEQ ID NO: 18 - protein, 13 aa, homo sapiens
AVIKTLEKLVNIE

SEQ ID NO: 19 - protein, 13 aa, homo sapiens
KTLEKLVNIETGT

SEQ ID NO: 20 - protein, 13 aa, homo sapiens
EKLVNIETGTGDA

SEQ ID NO: 21 - protein, 13 aa, homo sapiens
KLVNIETGTGDAE

SEQ ID NO: 22 - protein, 13 aa, homo sapiens
VNIETGTGDAEGI

SEQ ID NO: 23 - protein, 13 aa, homo sapiens
EGIAAAGNFLEAE

SEQ ID NO: 24 - protein, 13 aa, homo sapiens
GNFLEAELKNLGF

SEQ ID NO: 25 - protein, 13 aa, homo sapiens
NFLEAELKNLGFT

SEQ ID NO: 26 - protein, 13 aa, homo sapiens
AELKNLGFTVTRS

SEQ ID NO: 27 - protein, 13 aa, homo sapiens
KNLGFTVTRSKSA

SEQ ID NO: 28 - protein, 13 aa, homo sapiens
LGFTVTRSKSAGL

SEQ ID NO: 29 - protein, 13 aa, homo sapiens
FTVTRSKSAGLVV

SEQ ID NO: 30 - protein, 13 aa, homo sapiens
AGLVVGDNIVGKI

SEQ ID NO: 31 - protein, 13 aa, homo sapiens
GLVVGDNIVGKIK

SEQ ID NO: 32 - protein, 13 aa, homo sapiens
LVVGDNIVGKIKG

SEQ ID NO: 33 - protein, 13 aa, homo sapiens
DNIVGKIKGRGGK

SEQ ID NO: 34 - protein, 13 aa, homo sapiens
NIVGKIKGRGGKN

SEQ ID NO: 35 - protein, 13 aa, homo sapiens
GKIKGRGGKNLLL

SEQ ID NO: 36 - protein, 13 aa, homo sapiens
KNLLLMSHMDTVY

SEQ ID NO: 37 - protein, 13 aa, homo sapiens
NLLLMSHMDTVYL

SEQ ID NO: 38 - protein, 13 aa, homo sapiens
LLLMSHMDTVYLK

SEQ ID NO: 39 - protein, 13 aa, homo sapiens
LLMSHMDTVYLKG

SEQ ID NO: 40 - protein, 13 aa, homo sapiens
SHMDTVYLKGILA

SEQ ID NO: 41 - protein, 13 aa, homo sapiens
DTVYLKGILAKAP

SEQ ID NO: 42 - protein, 13 aa, homo sapiens
TVYLKGILAKAPF

SEQ ID NO: 43 - protein, 13 aa, homo sapiens
VYLKGILAKAPFR

SEQ ID NO: 44 - protein, 13 aa, homo sapiens
KGILAKAPFRVEG

SEQ ID NO: 45 - protein, 13 aa, homo sapiens
GILAKAPFRVEGD

SEQ ID NO: 46 - protein, 13 aa, homo sapiens
APFRVEGDKAYGP

SEQ ID NO: 47 - protein, 13 aa, homo sapiens
FRVEGDKAYGPGI

SEQ ID NO: 48 - protein, 13 aa, homo sapiens
KAYGPGIADDKGG

SEQ ID NO: 49 - protein, 13 aa, homo sapiens
PGIADDKGGNAVI

SEQ ID NO: 50 - protein, 13 aa, homo sapiens
NAVILHTLKLLKE

SEQ ID NO: 51 - protein, 13 aa, homo sapiens
AVILHTLKLLKEY

SEQ ID NO: 52 - protein, 13 aa, homo sapiens
VILHTLKLLKEYG

SEQ ID NO: 53 - protein, 13 aa, homo sapiens
HTLKLLKEYGVRD

SEQ ID NO: 54 - protein, 13 aa, homo sapiens
LKLLKEYGVRDYG

SEQ ID NO: 55 - protein, 13 aa, homo sapiens
KLLKEYGVRDYGT

SEQ ID NO: 56 - protein, 13 aa, homo sapiens
KEYGVRDYGTITV

SEQ ID NO: 57 - protein, 13 aa, homo sapiens
YGVRDYGTITVLF

SEQ ID NO: 58 - protein, 13 aa, homo sapiens
RDYGTITVLFNTD

SEQ ID NO: 59 - protein, 13 aa, homo sapiens
GTITVLFNTDEEK

SEQ ID NO: 60 - protein, 13 aa, homo sapiens
ITVLFNTDEEKGS

SEQ ID NO: 61 - protein, 13 aa, homo sapiens
TVLFNTDEEKGSF

SEQ ID NO: 62 - protein, 13 aa, homo sapiens
VLFNTDEEKGSFG

SEQ ID NO: 63 - protein, 13 aa, homo sapiens
GSFGSRDLIQEEA

SEQ ID NO: 64 - protein, 13 aa, homo sapiens
RDLIQEEAKLADY

SEQ ID NO: 65 - protein, 13 aa, homo sapiens
DLIQEEAKLADYV

SEQ ID NO: 66 - protein, 13 aa, homo sapiens
AKLADYVLSFEPT

SEQ ID NO: 67 - protein, 13 aa, homo sapiens
ADYVLSFEPTSAG

SEQ ID NO: 68 - protein, 13 aa, homo sapiens
DYVLSFEPTSAGD

SEQ ID NO: 69 - protein, 13 aa, homo sapiens
YVLSFEPTSAGDE

SEQ ID NO: 70 - protein, 13 aa, homo sapiens
LSFEPTSAGDEKL

SEQ ID NO: 71 - protein, 13 aa, homo sapiens
EKLSLGTSGIAYV

SEQ ID NO: 72 - protein, 13 aa, homo sapiens
LSLGTSGIAYVQV

SEQ ID NO: 73 - protein, 13 aa, homo sapiens
SGIAYVQVNITGK

SEQ ID NO: 74 - protein, 13 aa, homo sapiens
IAYVQVNITGKAS

SEQ ID NO: 75 - protein, 13 aa, homo sapiens
AYVQVNITGKASH

SEQ ID NO: 76 - protein, 13 aa, homo sapiens
VQVNITGKASHAG

SEQ ID NO: 77 - protein, 13 aa, homo sapiens
VNITGKASHAGAA

SEQ ID NO: 78 - protein, 13 aa, homo sapiens
PELGVNALVEASD

SEQ ID NO: 79 - protein, 13 aa, homo sapiens
LGVNALVEASDLV

SEQ ID NO: 80 - protein, 13 aa, homo sapiens
NALVEASDLVLRT

SEQ ID NO: 81 - protein, 13 aa, homo sapiens
ALVEASDLVLRTM

SEQ ID NO: 82 - protein, 13 aa, homo sapiens
SDLVLRTMNIDDK

SEQ ID NO: 83 - protein, 13 aa, homo sapiens
DLVLRTMNIDDKA

SEQ ID NO: 84 - protein, 13 aa, homo sapiens
LVLRTMNIDDKAK

SEQ ID NO: 85 - protein, 13 aa, homo sapiens
RTMNIDDKAKNLR

SEQ ID NO: 86 - protein, 13 aa, homo sapiens
MNIDDKAKNLRFN

SEQ ID NO: 87 - protein, 13 aa, homo sapiens
KNLRFNWTIAKAG

SEQ ID NO: 88 - protein, 13 aa, homo sapiens
LRFNWTIAKAGNV

SEQ ID NO: 89 - protein, 13 aa, homo sapiens
FNWTIAKAGNVSN

SEQ ID NO: 90 - protein, 13 aa, homo sapiens
WTIAKAGNVSNII

SEQ ID NO: 91 - protein, 13 aa, homo sapiens
GNVSNIIPASATL

SEQ ID NO: 92 - protein, 13 aa, homo sapiens
SNIIPASATLNAD

SEQ ID NO: 93 - protein, 13 aa, homo sapiens
NIIPASATLNADV

SEQ ID NO: 94 - protein, 13 aa, homo sapiens
ATLNADVRYARNE

SEQ ID NO: 95 - protein, 13 aa, homo sapiens
ADVRYARNEDFDA

SEQ ID NO: 96 - protein, 13 aa, homo sapiens
VRYARNEDFDAAM

SEQ ID NO: 97 - protein, 13 aa, homo sapiens
EDFDAAMKTLEER

SEQ ID NO: 98 - protein, 13 aa, homo sapiens
AAMKTLEERAQQK

SEQ ID NO: 99 - protein, 13 aa, homo sapiens
KTLEERAQQKKLP

SEQ ID NO: 100 - protein, 13 aa, homo sapiens
KKLPEADVKVIVT

SEQ ID NO: 101 - protein, 13 aa, homo sapiens
ADVKVIVTRGRPA

SEQ ID NO: 102 - protein, 13 aa, homo sapiens
VKVIVTRGRPAFN

SEQ ID NO: 103 - protein, 13 aa, homo sapiens
KVIVTRGRPAFNA

SEQ ID NO: 104 - protein, 13 aa, homo sapiens
VIVTRGRPAFNAG

SEQ ID NO: 105 - protein, 13 aa, homo sapiens
PAFNAGEGGKKLV

SEQ ID NO: 106 - protein, 13 aa, homo sapiens
KKLVDKAVAYYKE

SEQ ID NO: 107 - protein, 13 aa, homo sapiens
KLVDKAVAYYKEA

SEQ ID NO: 108 - protein, 13 aa, homo sapiens
KAVAYYKEAGGTL

SEQ ID NO: 109 - protein, 13 aa, homo sapiens
VAYYKEAGGTLGV

SEQ ID NO: 110 - protein, 13 aa, homo sapiens
AYYKEAGGTLGVE

SEQ ID NO: 111 - protein, 13 aa, homo sapiens
GTLGVEERTGGGT

SEQ ID NO: 112 - protein, 13 aa, homo sapiens
LGVEERTGGGTDA

SEQ ID NO: 113 - protein, 13 aa, homo sapiens
AAYAALSGKPVIE

SEQ ID NO: 114 - protein, 13 aa, homo sapiens
AALSGKPVIESLG

SEQ ID NO: 115 - protein, 13 aa, homo sapiens
KPVIESLGLPGFG

SEQ ID NO: 116 - protein, 13 aa, homo sapiens
PVIESLGLPGFGY

SEQ ID NO: 117 - protein, 13 aa, homo sapiens
ESLGLPGFGYHSD

SEQ ID NO: 118 - protein, 13 aa, homo sapiens
LGLPGFGYHSDKA

SEQ ID NO: 119 - protein, 13 aa, homo sapiens
PGFGYHSDKAEYV

SEQ ID NO: 120 - protein, 13 aa, homo sapiens
FGYHSDKAEYVDI

SEQ ID NO: 121 - protein, 13 aa, homo sapiens
AEYVDISAIPRRL

SEQ ID NO: 122 - protein, 13 aa, homo sapiens
EYVDISAIPRRLY

SEQ ID NO: 123 - protein, 13 aa, homo sapiens
VDISAIPRRLYMA

SEQ ID NO: 124 - protein, 13 aa, homo sapiens
SAIPRRLYMAARL

SEQ ID NO: 125 - protein, 13 aa, homo sapiens
RRLYMAARLIMDL

SEQ ID NO: 126 - protein, 13 aa, homo sapiens
RLYMAARLIMDLG

SEQ ID NO: 127 - protein, 13 aa, homo sapiens
LYMAARLIMDLGA

SEQ ID NO: 128 - protein, 15 aa, homo sapiens
QKRDNVLFQAATDEQ

SEQ ID NO: 129 - protein, 15 aa, homo sapiens
DNVLFQAATDEQPAV

SEQ ID NO: 130 - protein, 15 aa, homo sapiens
LFQAATDEQPAVIKT

SEQ ID NO: 131 - protein, 15 aa, homo sapiens
AATDEQPAVIKTLEK

SEQ ID NO: 132 - protein, 15 aa, homo sapiens
DEQPAVIKTLEKLVN

SEQ ID NO: 133 - protein, 15 aa, homo sapiens
PAVIKTLEKLVNIET

SEQ ID NO: 134 - protein, 15 aa, homo sapiens
IKTLEKLVNIETGTG

SEQ ID NO: 135 - protein, 15 aa, homo sapiens
LVNIETGTGDAEGIA

SEQ ID NO: 136 - protein, 15 aa, homo sapiens
IETGTGDAEGIAAAG

SEQ ID NO: 137 - protein, 15 aa, homo sapiens
GTGDAEGIAAAGNFL

SEQ ID NO: 138 - protein, 15 aa, homo sapiens
DAEGIAAAGNFLEAE

SEQ ID NO: 139 - protein, 15 aa, homo sapiens
GIAAAGNFLEAELKN

SEQ ID NO: 140 - protein, 15 aa, homo sapiens
AAGNFLEAELKNLGF

SEQ ID NO: 141 - protein, 15 aa, homo sapiens
NFLEAELKNLGFTVT

SEQ ID NO: 142 - protein, 15 aa, homo sapiens
EAELKNLGFTVTRSK

SEQ ID NO: 143 - protein, 15 aa, homo sapiens
LKNLGFTVTRSKSAG

SEQ ID NO: 144 - protein, 15 aa, homo sapiens
LGFTVTRSKSAGLVV

SEQ ID NO: 145 - protein, 15 aa, homo sapiens
TVTRSKSAGLVVGDN

SEQ ID NO: 146 - protein, 15 aa, homo sapiens
RSKSAGLVVGDNIVG

SEQ ID NO: 147 - protein, 15 aa, homo sapiens
SAGLVVGDNIVGKIK

SEQ ID NO: 148 - protein, 15 aa, homo sapiens
LVVGDNIVGKIKGRG

SEQ ID NO: 149 - protein, 15 aa, homo sapiens
GDNIVGKIKGRGGKN

SEQ ID NO: 150 - protein, 15 aa, homo sapiens
IVGKIKGRGGKNLLL

SEQ ID NO: 151 - protein, 15 aa, homo sapiens
KIKGRGGKNLLLMSH

SEQ ID NO: 152 - protein, 15 aa, homo sapiens
GRGGKNLLLMSHMDT

SEQ ID NO: 153 - protein, 15 aa, homo sapiens
GKNLLLMSHMDTVYL

SEQ ID NO: 154 - protein, 15 aa, homo sapiens
LLLMSHMDTVYLKGI

SEQ ID NO: 155 - protein, 15 aa, homo sapiens
MSHMDTVYLKGILAK

SEQ ID NO: 156 - protein, 15 aa, homo sapiens
MDTVYLKGILAKAPF

SEQ ID NO: 157 - protein, 15 aa, homo sapiens
VYLKGILAKAPFRVE

SEQ ID NO: 158 - protein, 15 aa, homo sapiens
KGILAKAPFRVEGDK

SEQ ID NO: 159 - protein, 15 aa, homo sapiens
LAKAPFRVEGDKAYG

SEQ ID NO: 160 - protein, 15 aa, homo sapiens
APFRVEGDKAYGPGI

SEQ ID NO: 161 - protein, 15 aa, homo sapiens
RVEGDKAYGPGIADD

SEQ ID NO: 162 - protein, 15 aa, homo sapiens
GDKAYGPGIADDKGG

SEQ ID NO: 163 - protein, 15 aa, homo sapiens
AYGPGIADDKGGNAV

SEQ ID NO: 164 - protein, 15 aa, homo sapiens
PGIADDKGGNAVILH

SEQ ID NO: 165 - protein, 15 aa, homo sapiens
ADDKGGNAVILHTLK

SEQ ID NO: 166 - protein, 15 aa, homo sapiens
KGGNAVILHTLKLLK

SEQ ID NO: 167 - protein, 15 aa, homo sapiens
NAVILHTLKLLKEYG

SEQ ID NO: 168 - protein, 15 aa, homo sapiens
ILHTLKLLKEYGVRD

SEQ ID NO: 169 - protein, 15 aa, homo sapiens
TLKLLKEYGVRDYGT

SEQ ID NO: 170 - protein, 15 aa, homo sapiens
LLKEYGVRDYGTITV

SEQ ID NO: 171 - protein, 15 aa, homo sapiens
EYGVRDYGTITVLFN

SEQ ID NO: 172 - protein, 15 aa, homo sapiens
VRDYGTITVLFNTDE

SEQ ID NO: 173 - protein, 15 aa, homo sapiens
YGTITVLFNTDEEKG

SEQ ID NO: 174 - protein, 15 aa, homo sapiens
ITVLFNTDEEKGSFG

SEQ ID NO: 175 - protein, 15 aa, homo sapiens
LFNTDEEKGSFGSRD

SEQ ID NO: 176 - protein, 15 aa, homo sapiens
TDEEKGSFGSRDLIQ

SEQ ID NO: 177 - protein, 15 aa, homo sapiens
EKGSFGSRDLIQEEA

SEQ ID NO: 178 - protein, 15 aa, homo sapiens
SFGSRDLIQEEAKLA

SEQ ID NO: 179 - protein, 15 aa, homo sapiens
SRDLIQEEAKLADYV

SEQ ID NO: 180 - protein, 15 aa, homo sapiens
LIQEEAKLADYVLSF

SEQ ID NO: 181 - protein, 15 aa, homo sapiens
EEAKLADYVLSFEPT

SEQ ID NO: 182 - protein, 15 aa, homo sapiens
KLADYVLSFEPTSAG

SEQ ID NO: 183 - protein, 15 aa, homo sapiens
DYVLSFEPTSAGDEK

SEQ ID NO: 184 - protein, 15 aa, homo sapiens
LSFEPTSAGDEKLSL

SEQ ID NO: 185 - protein, 15 aa, homo sapiens
EPTSAGDEKLSLGTS

SEQ ID NO: 186 - protein, 15 aa, homo sapiens
SAGDEKLSLGTSGIA

SEQ ID NO: 187 - protein, 15 aa, homo sapiens
DEKLSLGTSGIAYVQ

SEQ ID NO: 188 - protein, 15 aa, homo sapiens
LSLGTSGIAYVQVNI

SEQ ID NO: 189 - protein, 15 aa, homo sapiens
GTSGIAYVQVNITGK

SEQ ID NO: 190 - protein, 15 aa, homo sapiens
GIAYVQVNITGKASH

SEQ ID NO: 191 - protein, 15 aa, homo sapiens
YVQVNITGKASHAGA

SEQ ID NO: 192 - protein, 15 aa, homo sapiens
VNITGKASHAGAAPE

SEQ ID NO: 193 - protein, 15 aa, homo sapiens
TGKASHAGAAPELGV

SEQ ID NO: 194 - protein, 15 aa, homo sapiens
ASHAGAAPELGVNAL

SEQ ID NO: 195 - protein, 15 aa, homo sapiens
AGAAPELGVNALVEA

SEQ ID NO: 196 - protein, 15 aa, homo sapiens
APELGVNALVEASDL

SEQ ID NO: 197 - protein, 15 aa, homo sapiens
LGVNALVEASDLVLR

SEQ ID NO: 198 - protein, 15 aa, homo sapiens
NALVEASDLVLRTMN

SEQ ID NO: 199 - protein, 15 aa, homo sapiens
VEASDLVLRTMNIDD

SEQ ID NO: 200 - protein, 15 aa, homo sapiens
SDLVLRTMNIDDKAK

SEQ ID NO: 201 - protein, 15 aa, homo sapiens
VLRTMNIDDKAKNLR

SEQ ID NO: 202 - protein, 15 aa, homo sapiens
TMNIDDKAKNLRFNW

SEQ ID NO: 203 - protein, 15 aa, homo sapiens
IDDKAKNLRFNWTIA

SEQ ID NO: 204 - protein, 15 aa, homo sapiens
KAKNLRFNWTIAKAG

SEQ ID NO: 205 - protein, 15 aa, homo sapiens
NLRFNWTIAKAGNVS

SEQ ID NO: 206 - protein, 15 aa, homo sapiens
FNWTIAKAGNVSNII

SEQ ID NO: 207 - protein, 15 aa, homo sapiens
TIAKAGNVSNIIPAS

SEQ ID NO: 208 - protein, 15 aa, homo sapiens
KAGNVSNIIPASATL

SEQ ID NO: 209 - protein, 15 aa, homo sapiens
NVSNIIPASATLNAD

SEQ ID NO: 210 - protein, 15 aa, homo sapiens
NIIPASATLNADVRY

SEQ ID NO: 211 - protein, 15 aa, homo sapiens
PASATLNADVRYARN

SEQ ID NO: 212 - protein, 15 aa, homo sapiens
ATLNADVRYARNEDF

SEQ ID NO: 213 - protein, 15 aa, homo sapiens
NADVRYARNEDFDAA

SEQ ID NO: 214 - protein, 15 aa, homo sapiens
VRYARNEDFDAAMKT

SEQ ID NO: 215 - protein, 15 aa, homo sapiens
ARNEDFDAAMKTLEE

SEQ ID NO: 216 - protein, 15 aa, homo sapiens
EDFDAAMKTLEERAQ

SEQ ID NO: 217 - protein, 15 aa, homo sapiens
DAAMKTLEERAQQKK

SEQ ID NO: 218 - protein, 15 aa, homo sapiens
MKTLEERAQQKKLPE

SEQ ID NO: 219 - protein, 15 aa, homo sapiens
LEERAQQKKLPEADV

SEQ ID NO: 220 - protein, 15 aa, homo sapiens
RAQQKKLPEADVKVI

SEQ ID NO: 221 - protein, 15 aa, homo sapiens
QKKLPEADVKVIVTR

SEQ ID NO: 222 - protein, 15 aa, homo sapiens
LPEADVKVIVTRGRP

SEQ ID NO: 223 - protein, 15 aa, homo sapiens
ADVKVIVTRGRPAFN

SEQ ID NO: 224 - protein, 15 aa, homo sapiens
KVIVTRGRPAFNAGE

SEQ ID NO: 225 - protein, 15 aa, homo sapiens
VTRGRPAFNAGEGGK

SEQ ID NO: 226 - protein, 15 aa, homo sapiens
GRPAFNAGEGGKKLV

SEQ ID NO: 227 - protein, 15 aa, homo sapiens
AFNAGEGGKKLVDKA

SEQ ID NO: 228 - protein, 15 aa, homo sapiens
AGEGGKKLVDKAVAY

SEQ ID NO: 229 - protein, 15 aa, homo sapiens
GGKKLVDKAVAYYKE

SEQ ID NO: 230 - protein, 15 aa, homo sapiens
KLVDKAVAYYKEAGG

SEQ ID NO: 231 - protein, 15 aa, homo sapiens
DKAVAYYKEAGGTLG

SEQ ID NO: 232 - protein, 15 aa, homo sapiens
VAYYKEAGGTLGVEE

SEQ ID NO: 233 - protein, 15 aa, homo sapiens
YKEAGGTLGVEERTG

SEQ ID NO: 234 - protein, 15 aa, homo sapiens
AGGTLGVEERTGGGT

SEQ ID NO: 235 - protein, 15 aa, homo sapiens
TLGVEERTGGGTDAA

SEQ ID NO: 236 - protein, 15 aa, homo sapiens
VEERTGGGTDAAYAA

SEQ ID NO: 237 - protein, 15 aa, homo sapiens
RTGGGTDAAYAALSG

SEQ ID NO: 238 - protein, 15 aa, homo sapiens
GGTDAAYAALSGKPV

SEQ ID NO: 239 - protein, 15 aa, homo sapiens
DAAYAALSGKPVIES

SEQ ID NO: 240 - protein, 15 aa, homo sapiens
YAALSGKPVIESLGL

SEQ ID NO: 241 - protein, 15 aa, homo sapiens
LSGKPVIESLGLPGF

SEQ ID NO: 242 - protein, 15 aa, homo sapiens
KPVIESLGLPGFGYH

SEQ ID NO: 243 - protein, 15 aa, homo sapiens
IESLGLPGFGYHSDK

SEQ ID NO: 244 - protein, 15 aa, homo sapiens
LGLPGFGYHSDKAEY

SEQ ID NO: 245 - protein, 15 aa, homo sapiens
PGFGYHSDKAEYVDI

SEQ ID NO: 246 - protein, 15 aa, homo sapiens
GYHSDKAEYVDISAI

SEQ ID NO: 247 - protein, 15 aa, homo sapiens
SDKAEYVDISAIPRR

SEQ ID NO: 248 - protein, 15 aa, homo sapiens
AEYVDISAIPRRLYM

SEQ ID NO: 249 - protein, 15 aa, homo sapiens
VDISAIPRRLYMAAR

SEQ ID NO: 250 - protein, 15 aa, homo sapiens
SAIPRRLYMAARLIM

SEQ ID NO: 251 - protein, 15 aa, homo sapiens
PRRLYMAARLIMDLG

SEQ ID NO: 252 - protein, 15 aa, homo sapiens
LYMAARLIMDLGAGK

SEQ ID NO: 253 - protein, 15 aa, homo sapiens
AARLIMDLGAGKHHH

SEQ ID NO: 254 - protein, 15 aa, homo sapiens
LIMDLGAGKHHHHHH

SEQ ID NO: 255 - protein, 13 aa, homo sapiens
PKYVKQNTLKLAT

SEQ ID NO: 256 - protein, 15 aa, homo sapiens
KVVDQIKKISKPVQH

SEQ ID NO: 257 - DNA, 73 bp, Artificial Sequence
  Primer
cagaaacgtgacaacgttctgttccaggctgctaccgacgaacagccggctgttatcaaaaccctggaaaaac

SEQ ID NO: 258 - DNA, 81 bp, Artificial Sequence
  Primer
gaagttaccagcagcagcgataccttcagcgtcaccggtaccggtttcgatgttaaccagtttttccagggttttgataac

SEQ ID NO: 259 - DNA, 81 bp, Artificial Sequence
  Primer
gtatcgctgctgctggtaacttcctggaagctgaactgaaaaacctgggtttcaccgttacccgttctaaatctgctggtc

SEQ ID NO: 260 - DNA, 80 bp, Artificial Sequence
  Primer
cagcaggtttttaccaccacgacctttgattttaccaacgatgttgtcaccaacaaccagaccagcagatttagaacggg

SEQ ID NO: 261 - DNA, 73 bp, Artificial Sequence
  Primer
cgtggtggtaaaaacctgctgctgatgtctcacatggacaccgtttacctgaaaggtatcctggctaaagctc

SEQ ID NO: 262 - DNA, 73 bp, Artificial Sequence
  Primer
gtcgtcagcgatacccggaccgtaagctttgtcaccttcaacacggaacggagctttagccaggatacctttc

SEQ ID NO: 263 - DNA, 78 bp, Artificial Sequence
  Primer
gtccgggtatcgctgacgacaaaggtggtaacgctgttatcctgcacaccctgaaactgctgaaagaatacggtgttc

SEQ ID NO: 264 - DNA, 83 bp, Artificial Sequence
  Primer
gaaccgaaagaacctttttcttcgtcggtgttgaacagaacggtgatggtaccgtagtcacgaacaccgtattctttcagcag

SEQ ID NO: 265 - DNA, 76 bp, Artificial Sequence
  Primer
gaagaaaaaggttctttcggttctcgtgacctgatccaggaagaagctaaactggctgactacgttctgtctttcg

SEQ ID NO: 266 - DNA, 83 bp, Artificial Sequence
  Primer
ctgaacgtaagcgataccagaggtacccagagacagtttttcgtcaccagcagaggtcggttcgaaagacagaacgtagtcag

SEQ ID NO: 267 - DNA, 83 bp, Artificial Sequence
  Primer
ctctggtatcgcttacgttcaggttaacatcaccggtaaagcttctcacgctggtgctgctccggaactgggtgttaacgctc

SEQ ID NO: 268 - DNA, 80 bp, Artificial Sequence
  Primer
gtttttagctttgtcgtcgatgttcatggtacgcagaaccaggtcagaagcttcaaccagagcgttaacacccagttccg

SEQ ID NO: 269 - DNA, 76 bp, Artificial Sequence
  Primer
catcgacgacaaagctaaaaacctgcgtttcaactggaccatcgctaaagctggtaacgtttctaacatcatcccg

SEQ ID NO: 270 - DNA, 82 bp, Artificial Sequence
  Primer
catagcagcgtcgaagtcttcgttacgagcgtaacgaacgtcagcgttcagggtagcagaagccgggatgatgttagaaacg

SEQ ID NO: 271 - DNA, 76 bp, Artificial Sequence
  Primer
gaagacttcgacgctgctatgaaaaccctggaagaacgtgctcagcagaaaaaactgccggaagctgacgttaaag

SEQ ID NO: 272 - DNA, 64 bp, Artificial Sequence
  Primer
cttcaccagcgttgaaagccggacgaccacgggtaacgataactttaacgtcagcttccggcag

SEQ ID NO: 273 - DNA, 76 bp, Artificial Sequence
  Primer
cggctttcaacgctggtgaaggtggtaaaaaactggttgacaaagctgttgcttactacaaagaagctggtggtac

SEQ ID NO: 274 - DNA, 82 bp, Artificial Sequence
  Primer
gacagagcagcgtaagcagcgtcggtaccaccaccggtacgttcttcaacacccagggtaccaccagcttctttgtagtaag

SEQ ID NO: 275 - DNA, 52 bp, Artificial Sequence
  Primer
gctgcttacgctgctctgtctggtaaaccggttatcgaatctctgggtctgc

SEQ ID NO: 276 - DNA, 58 bp, Artificial Sequence
  Primer
cgtattcagctttgtcagagtggtaaccgaaacccggcagacccagagattcgataac

SEQ ID NO: 277 - DNA, 64 bp, Artificial Sequence
  Primer
cactctgacaaagctgaatacgttgacatctctgctatcccgcgtcgtctgtacatggctgctc

SEQ ID NO: 278 - DNA, 50 bp, Artificial Sequence
  Primer
tttaccagcacccaggtccatgatcagacgagcagccatgtacagacgac

SEQ ID NO: 279 - DNA, 37 bp, Artificial Sequence
  Primer
aaaaaagagctctttaccagcacccaggtccatgatc

SEQ ID NO: 280 - DNA, 40 bp, Artificial Sequence
  Primer
aaaaggcgcgccgcagaaacgtgacaacgttctgttccag

SEQ ID NO: 281 - DNA, 18 bp, Artificial Sequence
  Primer
ccgtttacataaaaggta

SEQ ID NO: 282 - DNA, 17 bp, Artificial Sequence
  Primer
acaccacgaaactgctg

SEQ ID NO: 283 - DNA, 18 bp, Artificial Sequence
  Primer
ccctgacactgctgaaag

SEQ ID NO: 284 - DNA, 18 bp, Artificial Sequence
  Primer
ctgaaaatgctgaaagaa

SEQ ID NO: 285 - DNA, 18 bp, Artificial Sequence
  Primer
gaaactgacgaaagaata

SEQ ID NO: 286 - DNA, 17 bp, Artificial Sequence
  Primer
acctgacccaggaagaa

SEQ ID NO: 287 - DNA, 18 bp, Artificial Sequence
  Primer
cgctctgactgaagcctc

SEQ ID NO: 288 - DNA, 18 bp, Artificial Sequence
  Primer
aagctggaaccgtttcta

SEQ ID NO: 289 - DNA, 18 bp, Artificial Sequence
  Primer
aacgtttctaccatcatc

SEQ ID NO: 290 - DNA, 17 bp, Artificial Sequence
  Primer
gtttctaacaccatccc

SEQ ID NO: 291 - DNA, 18 bp, Artificial Sequence
  Primer
taacatcaccccggcttc

SEQ ID NO: 292 - DNA, 18 bp, Artificial Sequence
  Primer
tctgctaccacgaacgct

SEQ ID NO: 293 - DNA, 18 bp, Artificial Sequence
  Primer
gtggtaaaacactggttg

SEQ ID NO: 294 - DNA, 18 bp, Artificial Sequence
  Primer
acaaagcatctgcttact

SEQ ID NO: 295 - DNA, 18 bp, Artificial Sequence
  Primer
tgttgctacctacaaaga

SEQ ID NO: 296 - DNA, 17 bp, Artificial Sequence
  Primer
ttgcttactccaaagaa

SEQ ID NO: 297 - DNA, 18 bp, Artificial Sequence
  Primer
taccttttatgtaaacgg

SEQ ID NO: 298 - DNA, 17 bp, Artificial Sequence
  Primer
cagcagtttcgtggtgt

SEQ ID NO: 299 - DNA, 18 bp, Artificial Sequence
  Primer
ctttcagcagtgtcaggg

SEQ ID NO: 300 - DNA, 18 bp, Artificial Sequence
  Primer
ttctttcagcattttcag

SEQ ID NO: 301 - DNA, 18 bp, Artificial Sequence
  Primer
tattctttcgtcagtttc

SEQ ID NO: 302 - DNA, 17 bp, Artificial Sequence
  Primer
ttcttcctgggtcaggt

SEQ ID NO: 303 - DNA, 18 bp, Artificial Sequence
  Primer
gaggcttcagtcagagcg

SEQ ID NO: 304 - DNA, 18 bp, Artificial Sequence
  Primer
tagaaacggttccagctt

SEQ ID NO: 305 - DNA, 18 bp, Artificial Sequence
  Primer
gatgatggtagaaacgtt

SEQ ID NO: 306 - DNA, 17 bp, Artificial Sequence
  Primer
gggatggtgttagaaac

SEQ ID NO: 307 - DNA, 18 bp, Artificial Sequence
  Primer
gaagccggggtgatgtta

SEQ ID NO: 308 - DNA, 18 bp, Artificial Sequence
  Primer
agcgttcgtggtagcaga

SEQ ID NO: 309 - DNA, 18 bp, Artificial Sequence
  Primer
caaccagtgttttaccac

SEQ ID NO: 310 - DNA, 18 bp, Artificial Sequence
  Primer
agtaagcagatgctttgt

SEQ ID NO: 311 - DNA, 18 bp, Artificial Sequence
  Primer
tctttgtaggtagcaaca

SEQ ID NO: 312 - DNA, 17 bp, Artificial Sequence
  Primer
ttctttggagtaagcaa